(19) United States
(12) Patent Application Publication
(10) Pub. No.: US 2003/0118491 A1
(43) Pub. Date: Jun. 26, 2003

(54) Filtered gas plasma sterilization container with improved circulation

(76) Inventors: Marcia A. Frieze, Alpine, NJ (US); Allan S. Frieze, Alpine, NJ (US); Ron Amster, New York, NY (US)

Correspondence Address: PRESTON GATES ELLIS & ROUVELAS MEEDS LLP, 1735 NEW YORK AVENUE, NW, SUITE 500, WASHINGTON, DC, 20006, US

(21) Appl. No.: 10/295,758

(22) Filed: Nov. 15, 2002

Related U.S. Application Data
(63) Continuation-in-part of application No. 09/622,153, filed on Aug. 11, 2000.
Application No. 09/622,153 is a continuation-in-part of application No. PCT/US98/17671, filed on Aug. 26, 1998.
(60) Provisional application No. 60/184,299, filed on Feb. 23, 2000.

(51) Int. Cl. A61L 2/08; A61L 2/00

(52) U.S. Cl. 422/297; 422/300; 422/292; 206/373

(57) ABSTRACT

The invention relates to a container and methods for sterilizing medical instruments with various embodiments including use of an instrument tray and a thermostatically controlled valve. In particular, this invention relates to a sterilization container useful for sterilization with various sterilization techniques including gas, gas plasma and steam. The sterilization container of the present invention permits steam or gas sterilant penetration and prevents microorganisms and dust from entering. The materials used may be made of a suitable metal, including an aluminum alloy, that may be heat treated and/or anodized or coated with a suitable material.

### CROSS REFERENCE TO RELATED APPLICATIONS

This application is a continuation-in-part application of co-pending U.S. application Ser. No. 09/622,153, filed 11 Aug. 2000; which is a continuation-in-part application of International Application No. PCT/US98/17671, filed 26 Aug. 1998, and published under PCT Article 21(2) in English; and claims the benefit of U.S. application Ser. No. 09/023,055, filed 12 Feb. 1998, now U.S. Pat. No. 5,968,459; and this application claims the benefit of U.S. application Ser. No. 09/622,150, filed 2 Oct. 2000, now U.S. Pat. No. 6,468,482; which is a continuation-in-part application of International Application No. PCT/US99/08049, filed 8 Apr. 1999, and published under PCT Article 21(2) in English; which is a continuation-in-part application of U.S. application Ser. No. 09/023,055, filed 12 Feb. 1998, now U.S. Pat. No. 5,968,459; and which is a continuation-in-part of International Application No. PCT/US98/17671, filed 26 Aug. 1998, and published under PCT Article 21(2) in English; and further, this application claims the benefit of pending U.S. application Ser. No. 10/070,621, filed 5 Mar. 2002, which is a continuation-in-part of International Application No. PCT/US01/04786, filed 14 Feb. 2001, and published under PCT Article 21(2) in English; and claims the benefit of provisional U.S. application Ser. No. 60/184,299, filed 23 Feb. 2000, abandoned; and this application claims the benefit of provisional U.S. Application Serial No. 60/333,814, filed 16 Nov. 2001, the entire contents and substance of which are hereby incorporated by reference.

### FIELD OF THE INVENTION

The invention relates to a container for sterilizing medical instruments with various embodiments. In particular, this invention relates to a sterilization container useful for sterilization with various sterilization techniques including gas, gas plasma and steam. Moreover, the sterilization container may have an instrument tray useful for flash sterilization and a thermostatically controlled valve. The sterilization container of the present invention permits steam or gas sterilant penetration and prevents microorganisms and dust from entering. The container of the present invention, including the container comprising the thermostatic valve, also allows storage of sterilized contents and reduces moisture within the sterilization container. The materials used may be made of a suitable metal, including, but not limited to, an aluminum alloy, that may be heat treated and/or anodized or coated with a suitable material.

### BACKGROUND OF THE INVENTION

1. Description of Related Art

Steam sterilization is a common method used for the sterilization of items, especially medical instruments, by processing the items in an autoclave and exposing them to high-pressure steam. This method requires the wrapping of individual items, heating the items with steam and then waiting for a drying/cooling period.

Sometimes, during surgical procedures commonly used instruments need to be quickly sterilized after use or inadvertent contamination, or on other emergency or “stat” bases. Under such circumstances the standard autoclave method would take too long. An alternative sterilization method, which can be used under these circumstances, is known as flash sterilization. During flash sterilization methods, metal instruments are not wrapped but rather, are heated directly by the steam allowing sterilization in a reduced period of time. One drawback to the use of flash sterilization is the lack of time for a drying period. When the items are still moist and hot from sterilization, microorganisms and dust can contaminate the items as they are transported from the autoclave/sterilizer. Nevertheless, flash sterilization results in reduced exposure time.

Healthcare staff members presently rapidly process whole instrument sets on a flash basis by using an open pan to flash the set. The open pan is then covered with a towel and transported to the Operating Room suite. There are infection control concerns with this process as the sterile contents can be contaminated in transport.

Some existing sealed container manufacturers recommend using their existing model by cracking open the lid of the container in the autoclave in order to flash sterilize. The container is open in the sterilizer to expedite the process and closed for transport. For example, Riley Medical (Auburn, Me.) manufactures the “Flash Pack, ™” a plastic sealed container designed for closed flash sterilization and transport. However, this model uses a pressure valve rather than a temperature valve in its design and is made of plastic. It has a pressure sensitive valve system that may stick and is very difficult to remove for cleaning. Decontamination and cleaning are crucial steps in the sterilization process. Wagner GmbH, Munich, Germany manufactures a sealed container validated for pre-vacuum steam only. It is not recommended for flash sterilization and the valve is very difficult to clean. Inspecting the container for contaminates is difficult as well. Neither the Riley container or the Wagner container can be stacked in the autoclave for storage. Both of these containers have a moisture problem because their valves open under pressure and close before sufficient drying time occurs.

One common design for containers for rapid or flash sterilization is described in U.S. Pat. Nos. 5,097,865 and 4,748,003. Such containers use valves which require greater than atmospheric pressures to open the valves and allow the high-pressure steam to enter the container but are closed under normal pressure conditions. This approach has a number of disadvantages. Such containers must be opened to allow the steam to escape, thus breaking the sterile field. Even if kept sealed, these containers cannot maintain the sterile field for longer than 24 hours. Also, the high temperature, high-pressure valves needed for this method are very complex and very expensive. In addition, such containers do not provide an indication as to whether or not the valve properly functioned to allow the high pressure steam to enter the container and pressure sensitive valves are very difficult to test for proper function.

Sterilization time can also be reduced by limiting the amount of instruments placed into the sterilization container. If too many instruments with too much mass are placed into the sterilization container, “stat” sterilization will not be effective. One method to restrict the amount of instruments placed into the sterilization container is to weigh the instruments. Weighing of instruments, however, can be time consuming in itself and difficult to control given the various sizes, shapes and odd surface areas of medical instruments.

In addition to steam sterilization the industry is beginning to use gas plasma as an alternative. One commercially available gas plasma system is sold as Sterrad® by Advanced Sterilization Products, a division of the Johnson & Johnson Company. Gas plasma has known advantages over steam sterilization, including sterilizing at a lower temperature than required for steam sterilization, which is beneficial when sterilizing temperature-sensitive devices. However, it has been learned that frequently the sterilizing gas plasma does not reach all important surfaces on the inside of the sterilization container, especially where long tubular instruments or cables are contained. Accordingly, there are believed to be very few, if any, sterilization containers approved for use with gas plasma. Clearly a technique is missing in the prior art to guarantee satisfactory circulation of gas plasma within a sterilization container, especially where it is critical to reach the edges and corners of the interior of the sterilization container and to penetrate internal components such as laparascopic guides and tubing. The present invention, however, maintains its efficacy when utilized with gas, gas plasma or steam as the sterilant.

### SUMMARY OF THE INVENTION

Briefly described, the invention comprises a sterilization container and a sterilization method for sterilizing items that mitigates the problems above and which may allow for extended, sterile storage of the sterilized items. The container and methods may be used for flash sterilization or regular sterilization utilizing gas, gas plasma or steam as the sterilant. The sterilization container basically has a pan, a cover, one or more filters for preventing dust and microorganisms from entering the container and contaminating the sterilized items, and optionally may comprise an instrument tray, and may also optionally comprise a thermostatically controlled valve that allows steam, gas, or gas plasma to enter and exit the container during the sterilization process. These containers are suitable for use in a wide variety of sterilization processes and with a wide variety of sterilants, such as gas, gas plasma and steam, and are also suitable for use in the flash sterilization process commonly used in surgical theaters. The sterilization containers of the present invention may be made from a wide variety of materials, including, but not limited to, metals and other materials that may be used under the sterilization condition desired. Such metals include, but are not limited to, aluminum, aluminum alloys, heat treated metals, or other metals that are thermoconductive, and in one embodiment, an anodized aluminum alloy.

One embodiment of the present invention provides a lid with a first set of vent holes, a filter means adjacent to the first set of vent holes, a bottom attachable to the lid. The bottom may comprise side walls, a base and at least one set of vent holes in the base. In another embodiment of the invention, a second set of vent holes may be present in the bottom. In still another embodiment of the invention, a second or more sets of vent holes may be present in the lid. In one embodiment, the container is adapted to provide passage for a sterilizing medium through out the container and the vent holes. In some embodiments of the present invention, a set of vent holes may include many holes that may be concentric.

In one embodiment of the invention, each set of vent holes may have a filter associated with it. In another embodiment of the invention, one or more of the sets of vent holes may have a thermostatic valve assembly associated with it. In embodiments in which filters are used, a wide variety of filter materials may be used, including, but not limited to, paper, hydrophobic, polypropylene polyolefin, non-woven, or synthetic filter materials. In embodiments in which a thermostatic valve assembly is used, the assembly may include a cover that covers the vent holes and a thermostatic valve that provides a channel between the inside and outside of the sterilization container for a sterilization medium. In some embodiments, gas or gas plasma may be used as the sterilizing medium. In other embodiments steam may be used as the sterilizing medium. If steam is used, the thermostatic valve may open or close at any suitable temperatures, including, but not limited to, between about 150 and about 225 degrees Fahrenheit. In embodiments in which gas or gas plasma is used, the valve may open or close at any suitable temperatures, including, but not limited to, between about 100 and about 150 degrees. The thermostatic valve may allow simplified cleaning and sterilization since thermostatic valves may be of simple construction and may not require disassembly. The valve may allow sterilized contents to be stored once the thermostatic valve is closed.

In another embodiment of the present invention, the lid and bottom of the container may have one or more dimples, projections, channels, impressions or similar feature that engages with dimples, projections, channels, impressions or similar feature of other containers for more secure stacking or for providing clearance for vent holes. The dimples, projections, channels, impressions or similar feature in the bottom of the container may also act as feet to provide clearance for the bottom of the container. The feet provide bottom clearance so that the flow of sterilant into the filter assemblies, or evaporation of moisture from inside the container, is not obstructed.

Some embodiments may include an instrument tray that fits inside the sterilization container. The instrument tray may have a base connected to one or more side walls or with one or more handles. In one embodiment, the tray may have two side walls with handles, or two end walls that are also handles. The handles may also provide clearance from the filter or valve assembly in the top of the container, as well as from additional trays inside the container once the container is closed. In another embodiment, there may be one or more handles and from none, one, two, three, four or more side walls. The instrument tray may also include a divider system that prevents the instruments from contacting each other. In some embodiments of the present invention, the divider system may be brackets; the brackets may be scalloped. In one embodiment, when the brackets are in use, the tray may have none, one, two, three, four or more side walls and none, one or more handles. The instrument tray may also have feet, and the feet may provide clearance from the container bottom, filter assemblies, or another tray inside the container.

In one embodiment of the present invention, the sterilization container may be made of aluminum or an aluminum alloy. In some embodiments the alloy may be heat-treated and/or anodized. In embodiments in which the alloy is heat treated, the heat treatment may be in a range of between about T4 and T6 temper. In one embodiment, the temper is about a T4, T5 or T6 temper. In embodiments in which the alloy is anodized the resulting coating may range from about 0.1 to about 0.5 mil. In other embodiments the coating may be about 0.2 or about 0.3 mil.

Another alternative embodiment of the invention, the container may have a means to insulate electrical contact between the lid and the bottom. In another embodiment, the container prevents excess internal moisture by allowing evaporation.

The present invention also provides a method for sterilization using the components described above. In one embodiment of the method for sterilization is performed by passing a sterilizing medium through a sterilization container in a sterilization chamber, and removing the container from the sterilization chamber.

These and other features of the invention may be more fully understood by reference to the following drawings.

### BRIEF DESCRIPTION OF THE DRAWINGS

FIG. 1, Panel A is a perspective view of one embodiment of the present invention.

FIG. 1, Panel B is a top perspective view of the flash sterilization container cover with a filter retainer.

FIG. 1, Panel C is a perspective view of one embodiment of the present invention with the top surface of the lid having a D-ring attached to it.

FIG. 2, Panel A is an exploded perspective view of one embodiment of the present invention.

FIG. 2, Panel B is a partial view of a cover opening configuration.

FIG. 3, Panel A is a side elevation view of one embodiment of the present invention.

FIG. 3, Panel B is a top plan view of one embodiment of the present invention with the locking means in the locked position.

FIG. 3, Panel C is a top plan view of one embodiment of the present invention with the locking means in the unlocked position.

FIG. 4, Panel A is a partial, side elevation view of a flash sterilization container cover with an incorporated filter.

FIG. 4, Panel B is a partial, side elevation view of a flash sterilization container and a filter cartridge.

FIG. 5, Panel A is an exploded view of an alternative embodiment of the present invention in which the base of the container includes two sets of circular vent holes.

FIG. 5, Panel B is a top plan view of the lid of the alternative embodiment illustrated in FIG. 5, Panel A.

FIG. 5, Panel C is an elevation cross-sectional view of the lid illustrated in FIG. 5, Panel B.

FIG. 5, Panel D is a partial, cross-sectional exploded view of the top, or first set, of circular vent means illustrated in a manner in which the pull ring is attached to the lid.

FIG. 5, Panel E is a detailed, cross-sectional end view of the lid of FIG. 5, Panel B illustrated in a manner in which the snap-on post or threaded screw-on assembly attaches to the filter retainer plate.

FIG. 5, Panel F illustrates the bottom of a gas plasma alternative embodiment with second and third sets of circular vent means in the bottom portion of the container.

FIG. 6, Panel A is an exploded view of another embodiment of the present invention illustrating a fourth set of circular vent means located adjacent to said first set of circular vent means in the lid of the container.

FIG. 6, Panel B is a top plan view of the lid of the alternative embodiment illustrated in FIG. 6, Panel A.

FIG. 6, Panel C is a side elevation cross-sectional view of the lid illustrated in FIG. 6, Panel B.

FIG. 6, Panel D is a partial detailed exploded view of the first set of circular vent means illustrating the manner in which the pull ring is attached to the lid.

FIG. 7, Panel A is a perspective view of the removable instrument tray that may be placed inside the container to keep instruments above any accumulated moisture in the container.

FIG. 7, Panel B is a side view of the removable instrument tray that may be placed inside the container to keep instruments above any accumulated moisture in the container.

FIG. 7, Panel C is a top plan view of the removable instrument tray that may be placed inside the container to keep instruments above any accumulated moisture in the container.

FIG. 7, Panel D is a side view of the removable instrument tray that may be placed inside the container to keep instruments above any accumulated moisture in the container.

FIG. 8, Panel A is a top plan view of the filter retainer with a thermostatic valve assembly and locking system.

FIG. 8, Panel B is a side view of the filter retainer with a thermostatic valve assembly

FIG. 8, Panel C depicts a valve retainer adapter for a thermostatic valve assembly.

FIG. 8, Panel D is a side view of a valve retainer adapter for a thermostatic valve assembly.

FIG. 8, Panel E depicts a valve retainer adapter for a thermostatic valve assembly.

FIG. 9, Panel A is a top view of one embodiment of an instrument tray used in conjunction with the present invention.

FIG. 9, Panel B is a side view of one embodiment of an instrument tray used in conjunction with the present invention

FIG. 10, Panel A is a top view of one embodiment of an instrument tray used in conjunction with the present invention.

FIG. 10, Panel B is a side view of one embodiment of an instrument tray used in conjunction with the present invention showing sides that are adapted to fold down.

FIG. 10, Panel C is a side view of one embodiment of an instrument tray with handles used in conjunction with the present invention.

FIG. 11, Panel A is a top view of one embodiment of an instrument tray used in conjunction with the present invention.

FIG. 11, Panel B is a side view of one embodiment of an instrument tray used in conjunction with the present invention.

FIG. 11, Panel C is a side view of one embodiment of an instrument tray with handles used in conjunction with the present invention.

FIG. 11, Panel D is a three-dimensional view of a tray assembly used in conjunction with the present invention.

FIG. 12, Panel A is a side view of one embodiment of the brackets for the instrument tray used in conjunction with the present invention.

FIG. 12, Panel B is a cut out from FIG. 12, Panel B depicting an individual bracket.

FIG. 13, Panel A is a side view of one embodiment of the brackets for the instrument tray used in conjunction with the present invention.

FIG. 13, Panel B is a cut out from FIG. 13, Panel B depicting an individual bracket.

### DETAILED DESCRIPTION OF THE INVENTION

It is to be understood that this invention is not limited to the particular methodology, protocols, filters, construction materials and sterilization media or sterilants described herein and as such may vary. It is also to be understood that the terminology used herein is for the purpose of describing particular embodiments only, and is not intended to limit the scope of the present invention, which will be limited only by the appended claims.

As used herein and in the appended claims, the singular forms “a,” “an,” and “the” include plural reference unless the context clearly indicates otherwise. Thus, for example, reference to a “filter” is a reference to one or more such filters and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications, which might be used in connection with the presently described invention. The publications discussed herein are provided solely for their disclosure prior to the filing date of the present application. Nothing herein is to be construed as an admission that the inventors are not entitled to antedate such disclosure by virtue of prior invention or for any other reason.

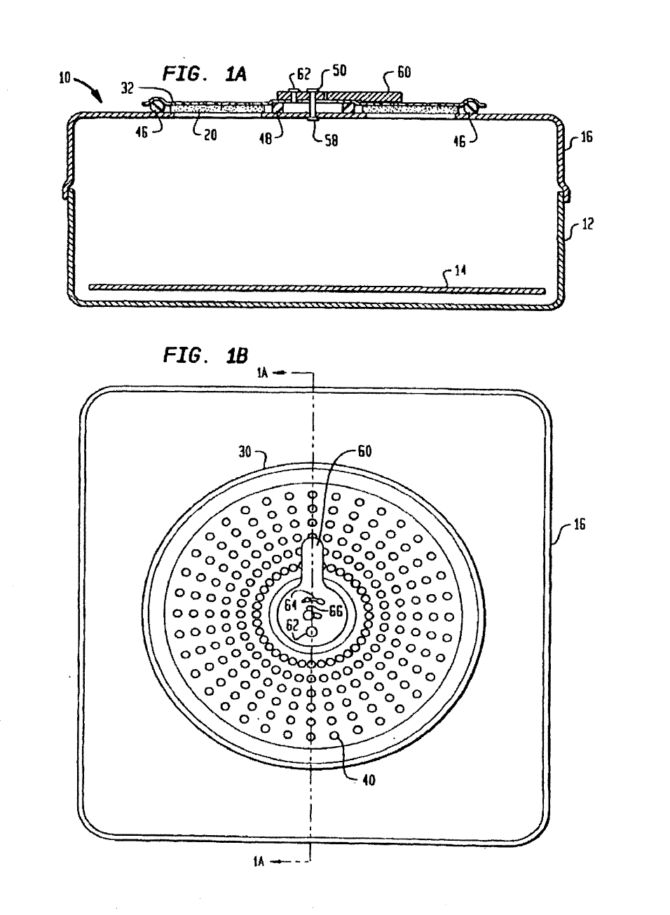

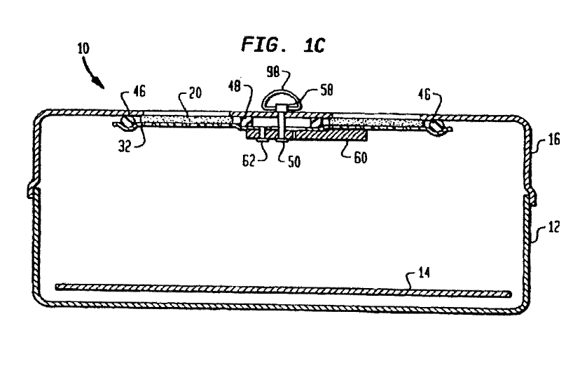

FIG. 1, Panel A depicts one embodiment of a sterilization container of the present invention. Sterilization container 10 may comprise a pan 12, which forms the bottom of the container that holds and supports a conventional sterilizable tray 14, and a cover 16, which forms the top of the container. The cover is preferably removably attached to the pan to form a hermetically sealed container. Hermetically sealing the container can be accomplished by conventional means such as hinges, clamps, and a sealing gasket. In addition, the sealing means may provide an audible report or signal, such as a snap or click, upon proper sealing of the container, which may provide the user with confirmation that the container is locked closed. The cover 16 is provided with an opening 18 at its top. This opening 18 may be covered with a filter 20 to allow steam or other sterilizing media to enter and exit the container through the opening by passing through a filter 20.

In one embodiment of the present invention, filter 20 can be removably or permanently attached to the cover. Filter 20 is made of a material, or combination of materials, such that the filter is permeable to the flow of steam or other sterilization mediums such as gas or gas plasma, for example, but will inhibit dust or other airborne particles or microorganisms from passing through. Examples of such materials are well known and commercially available and include, but are not limited to, paper; Teflon®, a registered trademark of E. I. Du Pont de Nemours and Co., Inc.; porous stainless steel; polysulfone; and a variety of hydrophobic materials, such as Gortex®, a registered trademark of W. L. Gore & Associates; and Kimguard® or Spunguard®, trademarks of the Kimberly-Clark Corporation. The filter is preferably attached to the cover by means that will prevent any steam, dust or other airborne particles or microorganisms from passing through the opening in the cover without passing through the filter.

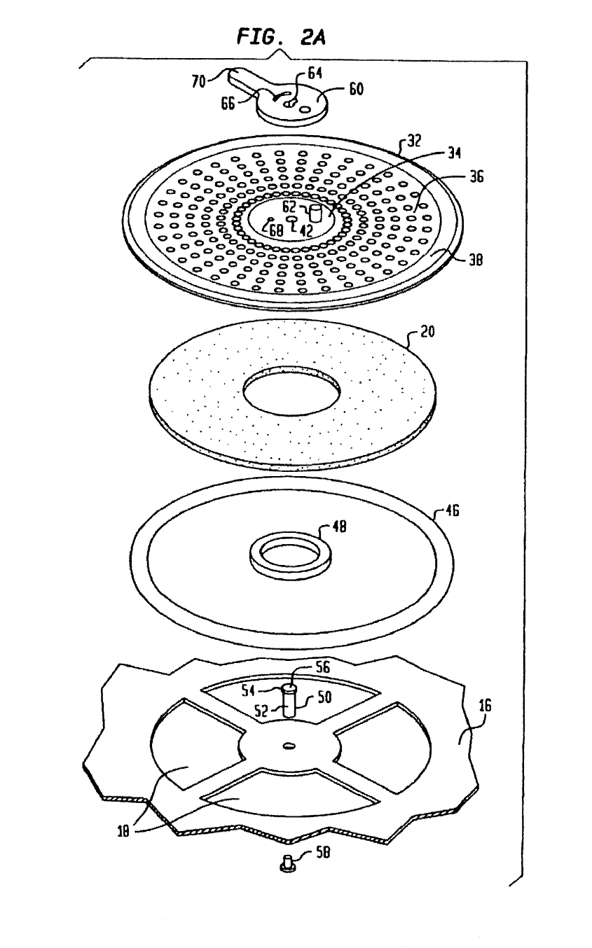

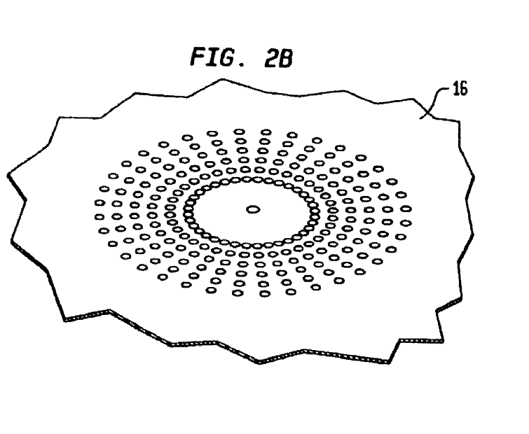

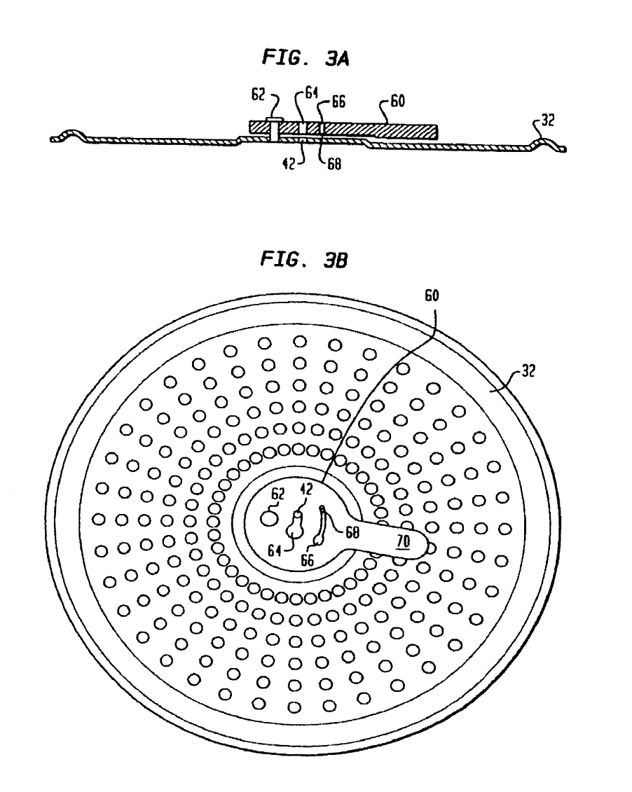

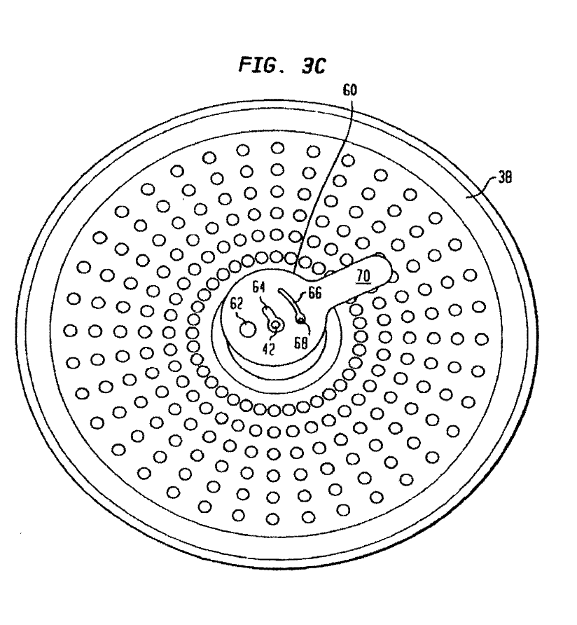

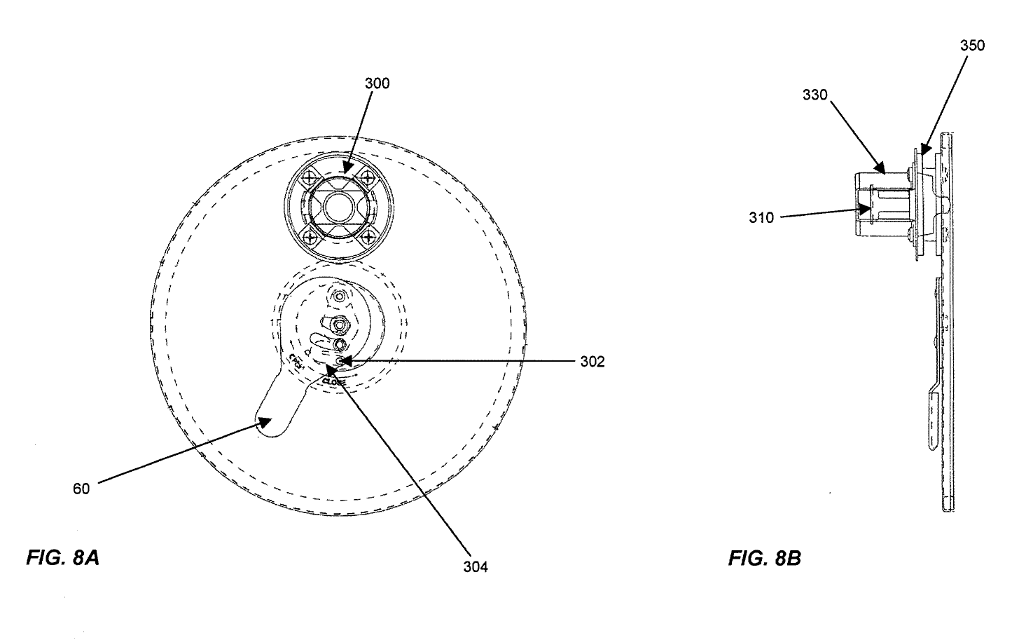

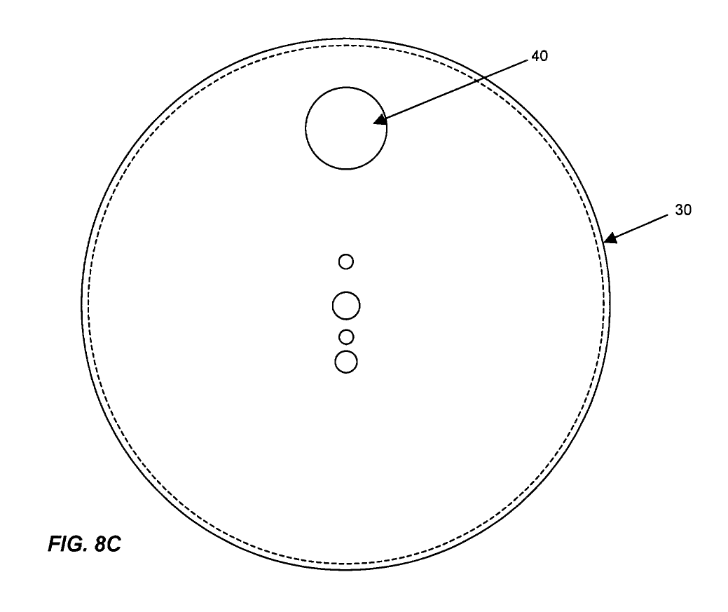

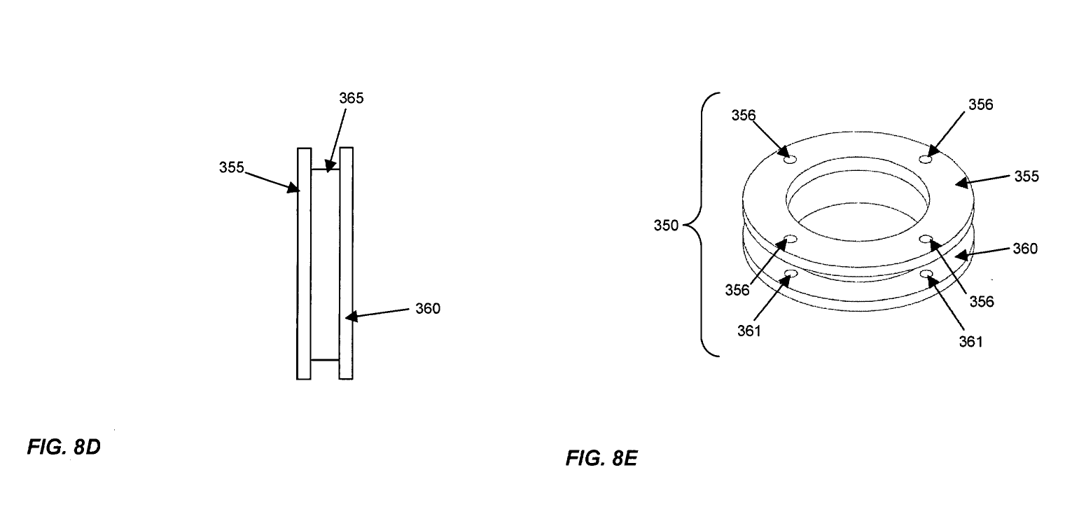

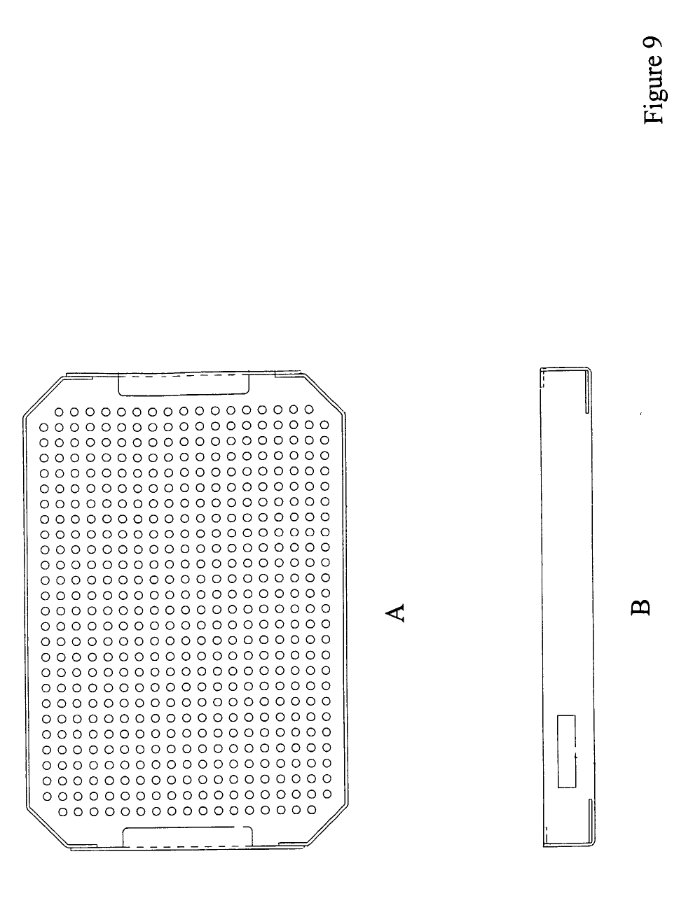

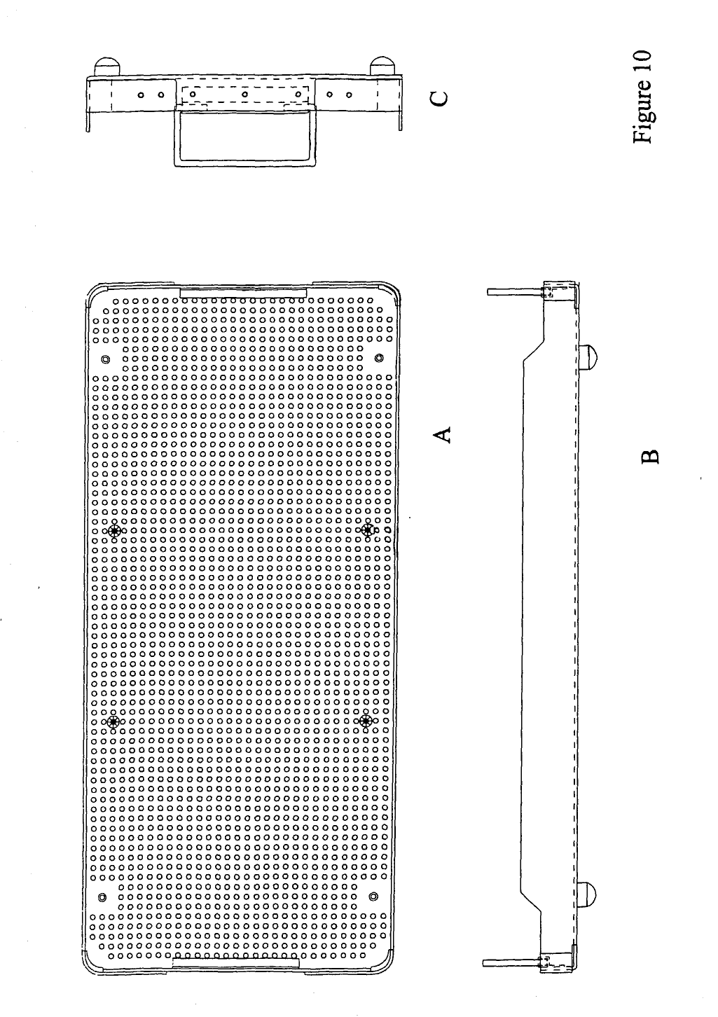

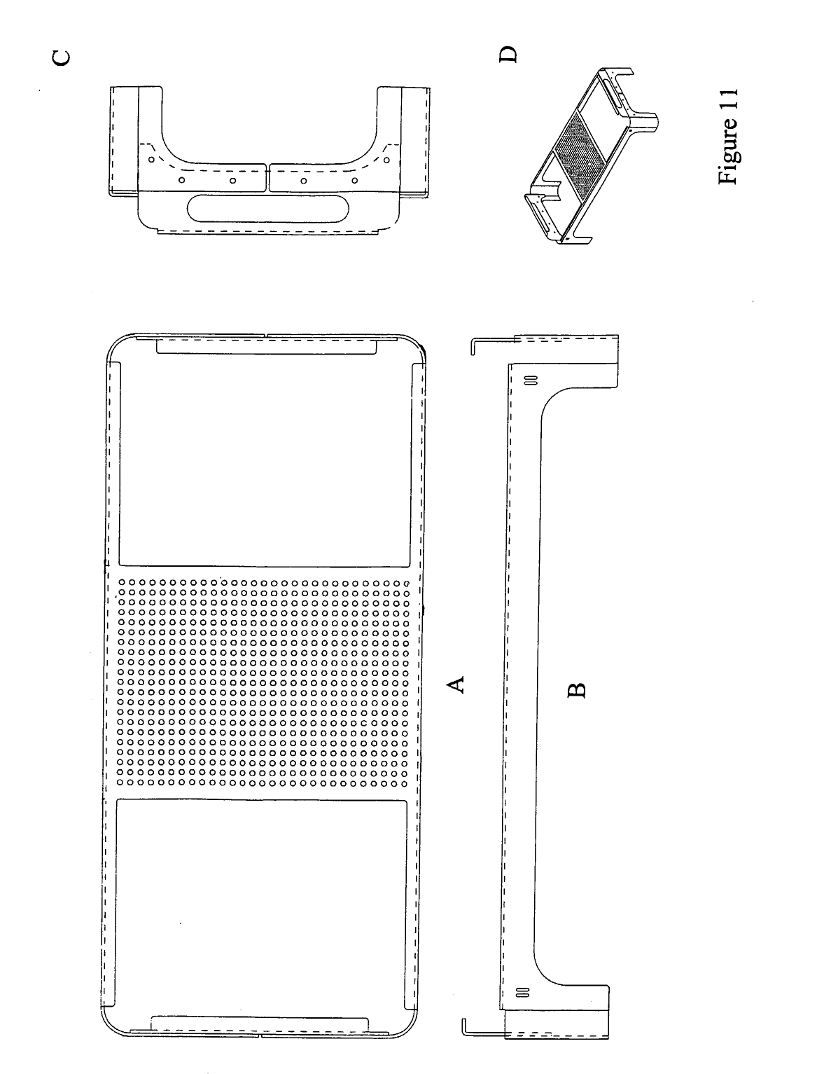

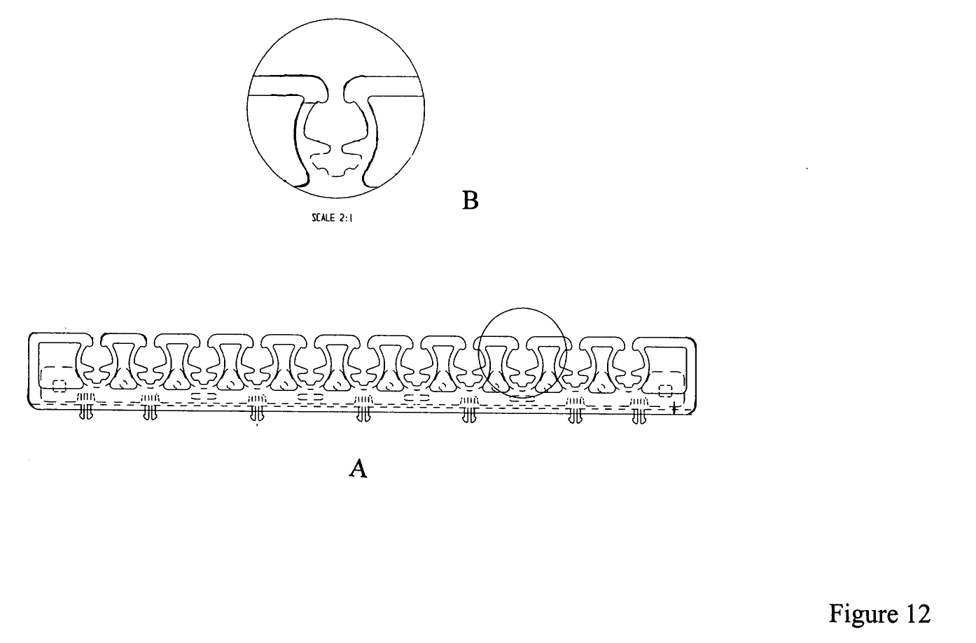

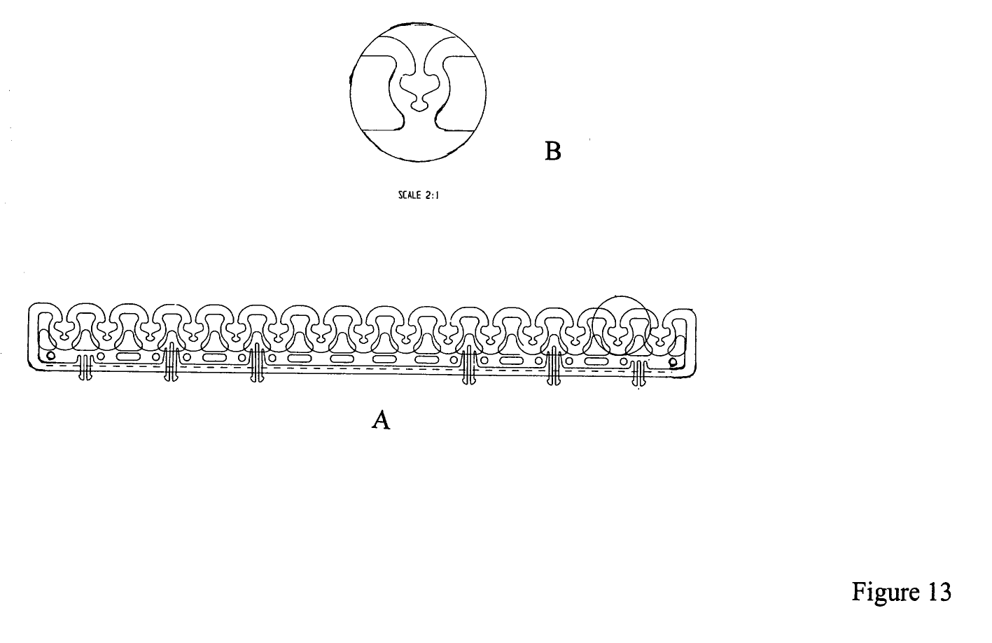

In a specific embodiment, the filter 20 is placed over the opening 18 in the top of the cover 16 and the filter is attached to the cover by a filter retainer 30. One embodiment of such a filter retainer is illustrated in FIG. 1, Panels A and B, FIG. 2, Panel A, and FIGS. 3, Panels A and B. Another embodiment of the present invention is depicted in FIG. 8, Panels A-C. Referring to FIG. 2, Panel A, the filter retainer 30 comprises a filter retainer disc 32 and a means for sealing the filter retainer disc to the cover. The filter retainer disc has an inner disc 34, a middle ring 36, and an outer ring 38. The middle ring 36 has an opening 40 to allow the flow of steam through the filter retainer disc 32, through the filter 20, and through the opening 18 in the cover. The filter retainer can have one or more sealing means for forming a seal between the filter and the cover. The outer ring 38 has a means for forming a seal between the filter and the cover. In one embodiment, the outer ring has an inverted U-shaped Cross-section. A gasket 46 may be placed in the inverted-U outer ring and can be made of silicone, neoprene, Teflon®, a registered trademark of E. I. Du Pont de Nemours and Co., Inc., or any other suitable material. Inner disc 34 may also have a sealing means if necessary, such as a gasket 48. Referring to FIG. 1, Panel B, the filter retainer may have an opening 40 to allow steam to pass from the outside the container to the inside of the container.

Referring back to FIG. 1, Panel C, a further alternative embodiment of the present invention may comprise a D-ring 98 attached to the end of a pin 50 connected to a cover 16. In this embodiment, the filter and filter retainer are mounted on the inside of the sterilization container. This arrangement permits D-ring 98 to be used as a handle to lift the cover without coming into contact with the side edges of the cover 16, thereby reducing the risk of contamination of the container contents.

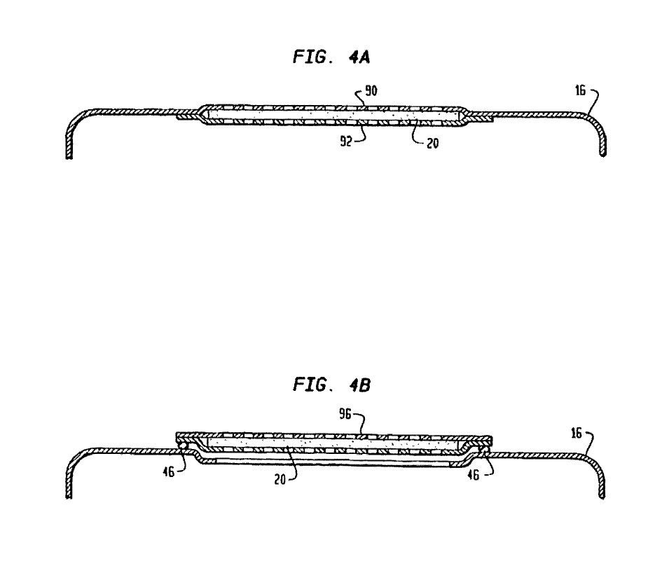

Alternative embodiments of the present embodiment may comprise having the filter manufactured as an integral part of the container as depicted in FIG. 4, Panel A, or having the filter incorporated into a self-contained removable filter unit or cartridge as depicted in FIG. 4, Panel B.

In an embodiment of the present invention, pin 50 acts to connect filter 20 to cover 16 and also engages a locking means associated with the cover. Pin 50 has sufficient length to extend from cover 16, through a locking means and past the top surface of the filter retainer 30 when the filter retainer is placed on the cover 16. Thus, the length of the pin body is essentially equal to the distance from the top surface of the cover to the top surface of the inner disc.

Referring to FIG. 2, Panel A, hole 42 in the inner disc engages the pin 50. Pin 50 is preferably located in the center of the opening 18 in the cover 16 and may be threaded for screw-on assembly. To locate the pin in the proper position, the opening in the cover can be, for example, a circular opening with cross pieces such as those illustrated in FIG. 2, Panel A.

Pin 50 can be made of separate elements attached by conventional means or preferably manufactured from a single piece of stock. Pin 50 may be comprised of a cylindrical body 52 having opposite ends and an outside diameter essentially equal to the inside diameter of the hole 42 in the filter retainer disc. Pin 50 may have a cylindrical neck 54 with a diameter smaller than the diameter of pin body 52 and equal to the width of the arc shaped slot 64 of the locking means. Pin 50 may also have a cylindrical head 56 having a top and a bottom and having a diameter larger than the pin neck 54, preferably equal to the diameter of the pin body 52. One end of the pin body 52 is attached to the cover 16 by conventional means, such as, a rivet 58, a screw, a thread, or a spot weld. Pin neck 54 may be attached to the end of the pin body 52 opposite the attachment to the cover and its length is preferably at least equal to the thickness of the sliding plate. The bottom of the pinhead is preferably attached to pin neck 54 at the end opposite the pin neck's attachment to pin body 52. The combined length of the pin body and pin neck is such that the bottom of the pin head is slightly lower than the top surface of the sliding plate 60.

Sliding plate 60 is positioned so that the end of the arc-shaped slot 64 having an increased width is aligned with the hole in the inner disc 42. Next, the filter retainer 30 is then placed over the opening in the cover 16 so that the retainer pin 50 passes through the hole in the inner disc 42 and the enlarged end of the arc-shaped slot 64. Sliding plate 60 is then rotated so that the arc-shaped slot 64 engages the pin neck 54, thereby preventing the pin from passing back through the arc-shaped slot and thus attaching the filter retainer to the cover.

When the arc-shaped pin slot 64 engages the pin neck 54, the filter retainer disc 32 will be forced toward the cover, compressing the gaskets 46, 48, and creating a seal between the filter retainer 30 and the cover 16. The top of pin head 56 can have a taper to facilitate the insertion of the retainer pin 50 through the hole in the filter retainer disc 42 and arc-shaped slot in the sliding plate 60. In an alternate embodiment, the opening in the cover can be comprised of a multiplicity of smaller openings in the cover, as depicted in FIG. 2, Panel B.

In a specific embodiment depicted in FIG. 1, Panel B, the filter retainer 30 has a means for limiting the rotation of the sliding plate 60 and facilitating the positioning of the sliding plate in an “open” position. In the open position, the enlarged end of the arc-shaped slot 64 (FIG. 2, Panel A) lines up with the hole in the inner disc 42. In a “locked” position, the opposite end of the arc-shaped slot lines up with the hole in the inner disc 42. One embodiment of a limiting means incorporates an arc-shaped limiting slot 66 on the sliding plate 60. The arc of the limiting slot 66 is preferably parallel to the arc of the arc-shaped slot 64 and has an effective radius larger than the radius of the arc-shaped slot 64. A locating pin 68 may be attached to, and extend from, the inner disc 34 such that it engages one end of the limiting slot 66 when the sliding plate 60 is in the locked position and engages the opposite end of the arc-shaped slot when the sliding plate is in the unlocked position. The locating pin 68 is preferably hemispherical-shaped to facilitate the movement of the sliding plate 60 over the locating pin 68. A hemispherical locating pin 68 can be made for example by inserting and attaching a ball bearing in a hole in the inner disc 34. The width of the limiting slot 66 is slightly less than the diameter of the locating pin 68. The width of the limiting slot 66 at each of the two, opposite ends, is enlarged slightly, forming two holes each having a diameter slightly larger than the diameter of the locating pin 68. Consequently, the sliding plate 60 may be held in the locked and open positions when the locating pin 68 engages each of the holes in the ends of the limiting slot 66, requiring the application of an external force to move the sliding plate between the two positions. In one embodiment, the locking mechanism produces an audible signal, such as a snap or click, when the lock is engaged, providing the user with confirmation that the assembly is locked closed.

As shown in FIG. 2, Panel A, the sliding plate 60 preferably has a handle 70 to facilitate moving the plate between the open and closed positions. The handle 70 preferably extends parallel to the plane of the sliding plate. The handle 70 can be attached to the sliding plate or manufactured with the sliding plate as a single piece.

FIG. 8, Panel A depicts an optional secondary locking mechanism. The mechanism may secure the filter cover to the positioning post and may operate in conjunction with sliding plate 60. A ball bearing 302, a bump, or an embossed dome engages the sliding plate 60 by means of an opening or hole 304 in the sliding plate. If a ball bearing is used, the ball bearing 302 pops into place in the closed position to provide a secondary positive lock. In one embodiment, the locking mechanism produces an audible signal, such as a snap or click, when the lock is engaged, providing the user with confirmation that the assembly is locked closed.

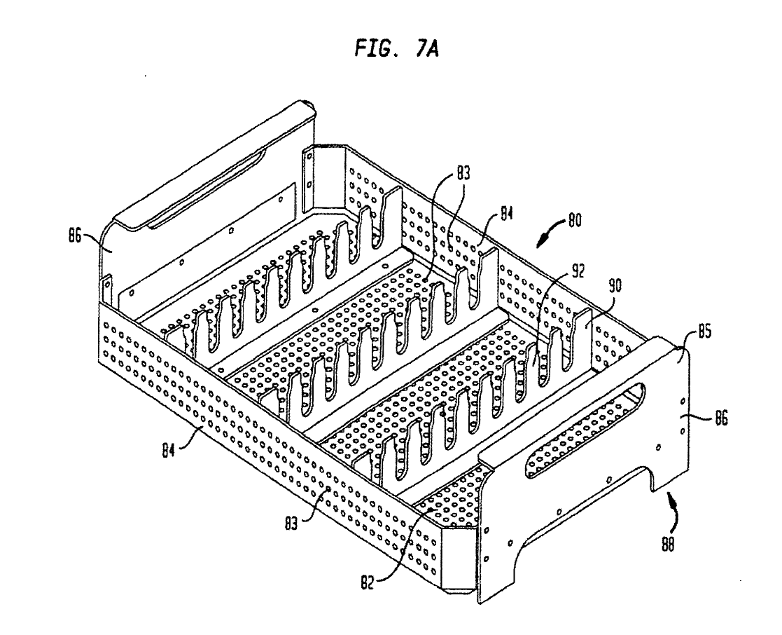

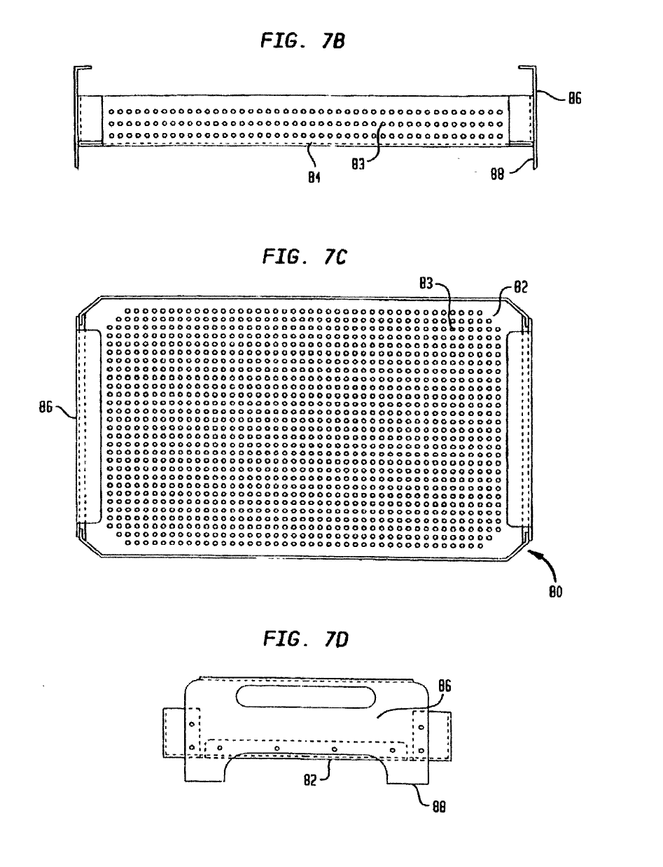

In one or more embodiments of the present invention, the sterilization container may hold an instrument tray. A removable instrument tray 80 is depicted in FIG. 7, Panel A and may be constructed of the same material as the container. The removable instrument tray is preferably placed inside the container. The tray has sides 84 to keep instruments from sliding out and has openings 83 on the sides 84 and in the bottom 82 to allow steam or plasma to pass through the tray. In one embodiment, the tray has no sides, only one or more handles attached to the base. In another embodiment, the tray has one or more side walls, wherein one or more of the side walls has one or more handles. In yet another embodiment, the tray has none, one or more side walls with no handles. In still another embodiment, the tray has sides that may be folded down or away to provide a tray without sides. The tray may be fitted with partitions 90 with any desired number of slots 92 which may hold instruments stationary and may prevent the items in the container from contacting one another. Additionally, partitions 90 and slots 92 may also prevent overloading of the tray in situations in which flash sterilization is needed. In one embodiment, the number of slots in the partitions only allows a fixed number of instruments to be placed in the tray at one time. In another embodiment, when the partitions are used, the tray has no sides, but only a base and, optionally, one or more handles. In a specific embodiment, thermostatic valve 310 of thermostatic valve assembly 300 may also act as a barrier or stop to prevent overloading of tray 80. Handles 85 may be attached to or cut into the ends 86 of tray 84 to allow for easy removal of the entire tray without the need to handle the instruments and may be formed inward to allow for aseptic removal of contents. Handles 85 also provide a stop to ensure clearance space for the inside of cover 16. Sides 86 or base 82 of tray 80 are fabricated with legs and extended to raise the entire tray 88 above the bottom of the container 16 and away from any moisture which may condense and collect in the bottom of the container. The handles of the tray, as well as the legs of the tray, may be arranged such that they limit the amount of instruments or material that may be placed in the tray at one time. The handles and legs of the tray may also be arranged to permit stacking of two or more trays in the container at one time for sterilization. Sides 86 may be constructed of the same material as the container or may be coated with a non-conductive or noncorrosive coating.

Although the foregoing embodiments are suitable for use in a wide variety of sterilization processes, such as standard steam-, gas- or gas plasma-based processes, the foregoing embodiments are also suitable for use in a flash sterilization environment using steam as a sterilizing medium. Gas plasma as a sterilization medium is commercially available from, among others, Advanced Sterilization Products, a division of Johnson & Johnson, under the trademark Sterrad®. Gas plasma, unlike higher temperature steam, can be used with a number of modem tools, such as cannulas, lumens, scopes, fiber optic cables, and cameras, without damaging them.

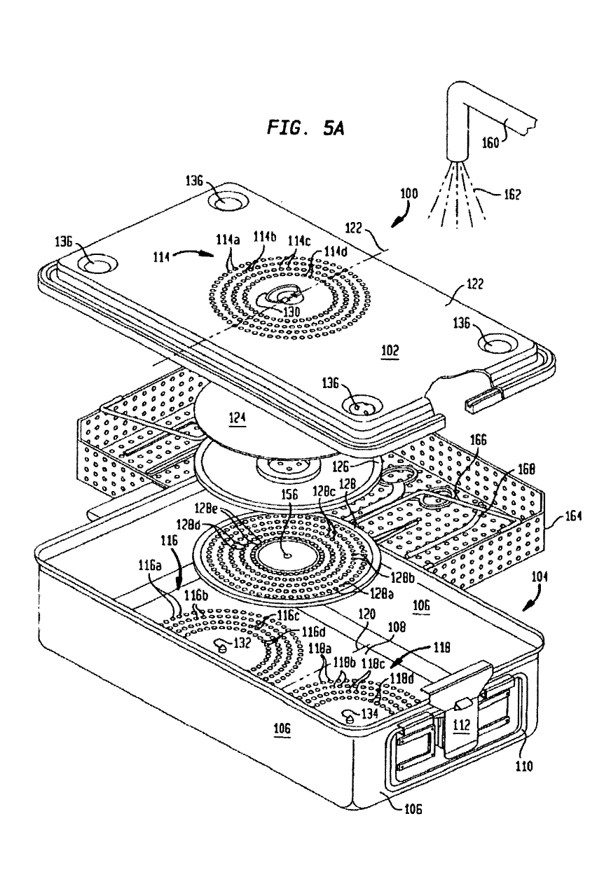

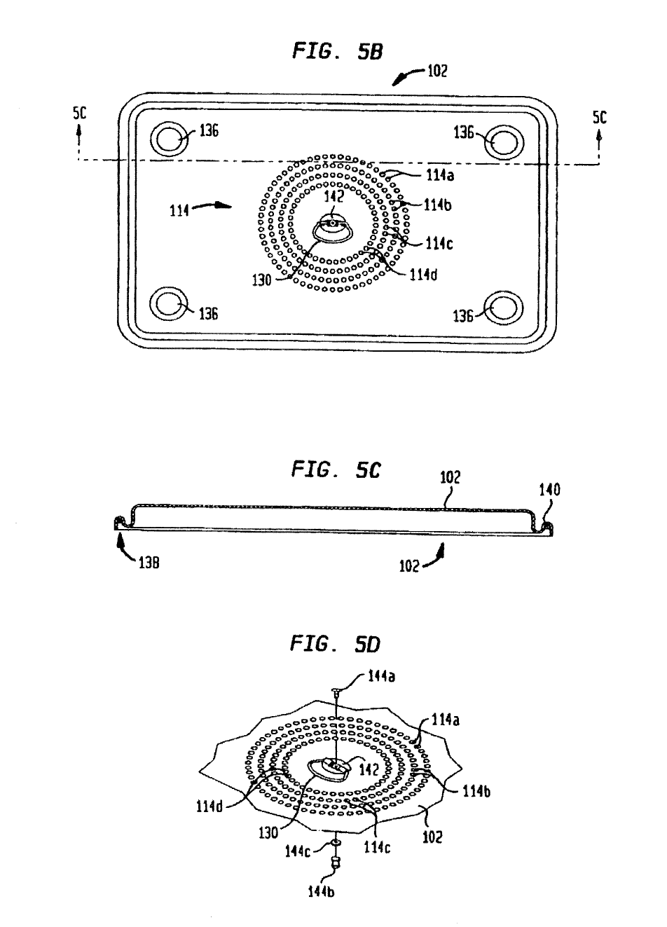

An embodiment 100 of the sterilization container of the present invention that also is suitable for use with gas or gas plasma is illustrated in the exploded view of FIG. 5, Panel A. The sterilization container includes a top or lid 102 that sits on top of a bottom or pan 104. Bottom 104 includes four sidewalls 106 and a bottom or base 108. A pair of wire handles, or bales 110 are located on opposite ends of the bottom portion 104 and are held in place by a pair of lockable latches 112.

A first set of vent holes 114 is located in top 102. Any desired arrangement or shape of vent holes may be utilized in the container of the present invention. The vent holes 114 are, in one embodiment, arranged as a group of four concentric circles with holes 114a, 114b, 114c and 114d in each, respectively. In all, the total number of holes in this embodiment may range from 1 to about 500 or more and have any desired size or shape. In one embodiment, the vent holes are a size that ranges in diameter from, but not limited to, about 3/16 inches to about 5/16 inches. The first set of vent holes 114 is located in one embodiment on the central axis 122 of the short dimension of the lid 102. The first set of vent holes 114 allows the sterilizing medium 162 to pass into the container. A pull ring 130, attached to a base 142 sits in the middle of the first set of vent holes 114 and is connected there by rivet assembly 144a, 144b, and 144c as shown in exploded detail in FIG. 5, Panel D. Lid 102 may also include one or more, or in one embodiment, four projections, channels, impressions or 136 which are adapted to engage with complimentary projections, channels, impressions or dimples in the base 108 (not shown) so that the containers 100 can be securely stacked on each other and permit circulation of steam, gas or gas plasma throughout the container. Additionally, projections, channels, impressions or dimples 108 provide clearance for vents in the bottom of the sterilization container so that the vents are not obstructed. The dimples may also be used to stack sterilized containers for storage, including embodiments of the present invention in which a thermostatic valve assembly 300 is used. Moreover, the dimples help to stabilize individual containers that are stacked for storage or transport.

A second set of vent holes 116 and optionally, in another embodiment, a third or more, as desired, sets of vent holes 118 are located in the base 108. In one embodiment, vent holes are placed on symmetrical opposite sides of center line 120 which represents the minor axis of the base 108. Any desired number of vent holes may be arranged on the base of the container in any desired pattern or shape. In all, the total number of holes in this embodiment may range from 1 to about 500 or more and have any desired size or shape. In one embodiment, the vent holes are a size that ranges in diameter from, but not limited to, about 3/16 inches to about 5/16 inches. The second set of vent holes 116 may, in one embodiment, also comprise four concentric circles having holes 116a, 116b, 116c and 116d, which may optionally also have the same or a different range of dimensions as the first set of vent holes 114. A hold-down stud 132 may be located in the center of the concentric circles and is intended to make, for example, a snap fit or screw-on fit with the retainer plate for the hydrophobic filter that goes there between. Similarly, the third or additional sets of vent holes 118 may, in one embodiment, optionally comprise four sets of concentric circles having holes 118a, 118b, 118c and 118d therein. A central post or stud 134 may also be located in the middle thereof and adapted, for example, to snap fit or screw-on fit into and may engage a filter retainer plate in the manner previously described. A circular filter disk 124, a hold down ring 126, and a perforated filter retainer plate 128 may be associated with a set of vent holes, such as first set of vent holes 114. A central hole 156 in the retainer plate 128 may snap fit or screw-on fit into and engages a stud 146 in the container, as illustrated in FIG. 5, Panel E. Similar sets of hydrophobic filters, rings, and retainer plates may be associated with other sets of vent holes, such as sets 116 and 118, as illustrated in FIG. 5, Panel F. Hydrophobic filters 124 are in one embodiment utilized when steam, gas or gas plasma acts as the sterilizing medium. In another embodiment, cellulosic paper filters, polypropylene polyolefin non-woven filters, or synthetic materials that are hydrophobic and capable of serving as a filter may be used when steam acts as the sterilizing medium. In embodiments using gas or gas plasma as a sterilizing medium, polyproylene polyolefin non-woven filters, or synthetic materials that are hydrophobic and capable of serving as a filter may be used. The Tyvek®, a trademark of E. I. du Pont de Nemours & Company, brand of polyethylene/polypropylene spun fiber is acceptable, as is Kimguard® and Spunguard®, trademarks of the Kimberly-Clark Corporation. In addition, hydrophobic filters do not absorb water, which allows for a quicker drying time.

The concentric holes 128a, 128b, 128c, 128d and 128e, are in one embodiment offset from the holes 114a, 114b, 114c, and 114d so as to the prevent “strikethrough,” i.e., to prevent sharp objects from entering the holes 114a, 114b, 114c and 114d and exiting through 128a, 128b, 128c, 128d or 128e.

As illustrated in FIG. 5, Panel C, lid 102 may include a groove 138 which may retain a gasket 140 which sits on top of the upper lip 150 of the bottom or base pan 104 as shown in FIG. 5, Panel F. FIG. 5, Panel F also shows in further detail how the bottom perforated retainer plate 152 attaches to the bottom stud 132 and keeps a hydrophobic filter in place above the second set of vent holes 116. Similarly, FIG. 5, Panel F also illustrates how another perforated filter retainer plate 154 engages, for example, a snap-on or screw-on stud 134 to hold another hydrophobic filter in place above the third set of perforated vent holes 118.

In an embodiment utilizing steam, gas or sterilant gas plasma as the sterilant may enter the sterilization container through the vent holes 114, pass through the hydrophobic filter 124 and emerge through the perforated base plate 128. In other embodiments, the steam, gas or gas plasma sterilant may enter through vent holes 114 and passes through thermostatic valve assembly 300, described infra. Because the top vent holes 114 are not located directly above the bottom vent hole or holes 116 and 118 the steam, gas or gas plasma is forced to migrate, and become somewhat turbulent as it attempts to find an exit through the second and/or third set of circular vent holes 116 and 118, respectively. This forces the steam, gas or gas plasma to more thoroughly mix and contact medical instruments or the like inside of the sterilization container and also forces the steam, gas or gas plasma further towards the comers and edges of the container. In alternative embodiments, the steam, gas or gas plasma may enter through one or more sets of vent holes and exit through one or more sets of vent holes. In one embodiment, the lid may contain one or many vent holes over a portion of, or the entire surface of, the lid. In another embodiment, the bottom may contain one or many vent holes over a portion of, or the entire surface of, the bottom. The present invention with the offset sets of vent holes works in all methods of sterilization, including, but not limited to, flash sterilization, steam sterilization, gas sterilization and gas plasma sterilization.

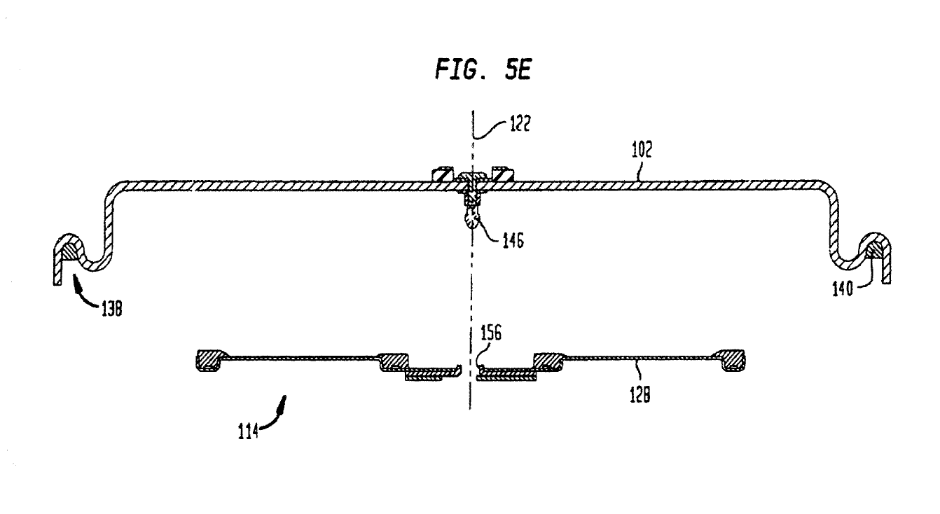

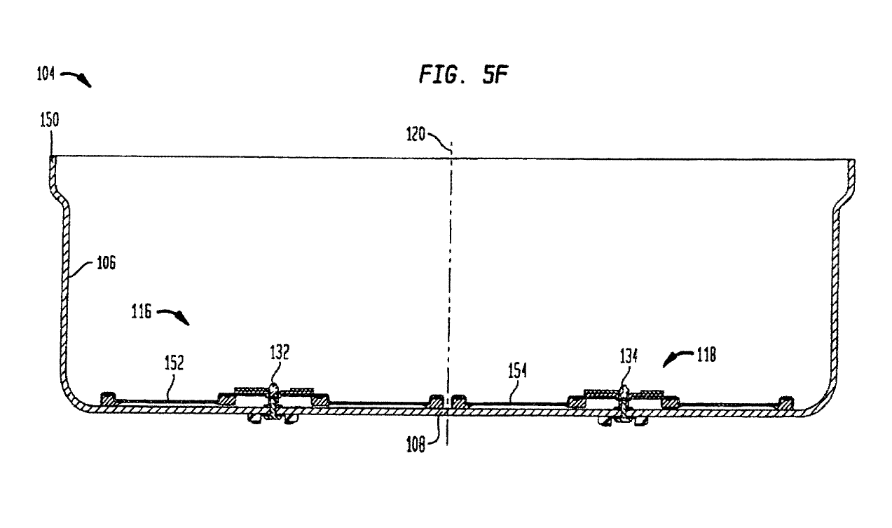

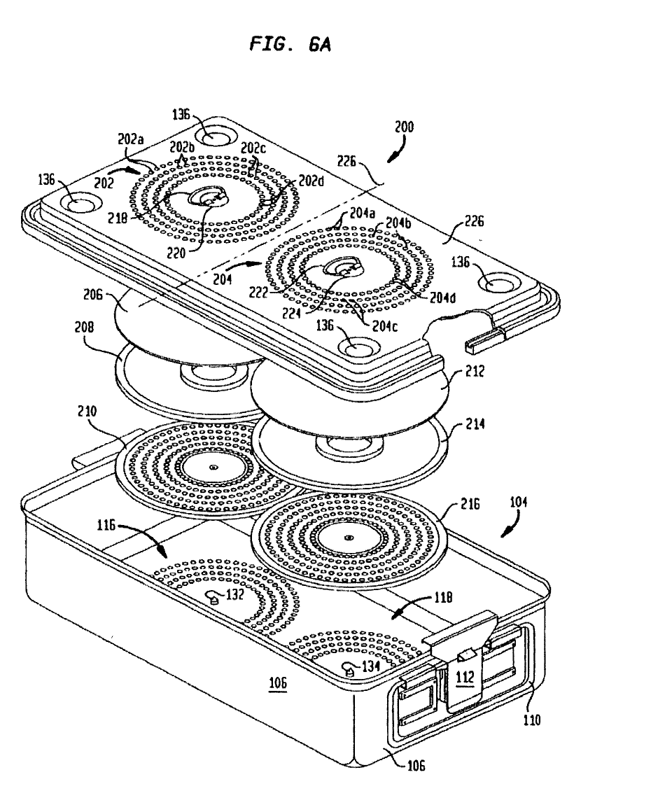

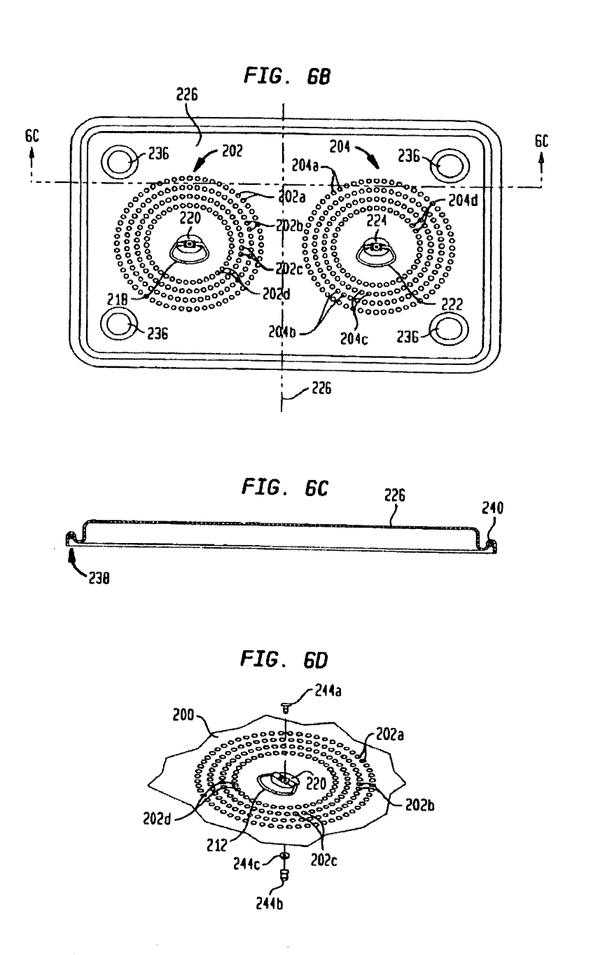

Another embodiment 200 of the present invention is illustrated in an exploded view shown in FIG. 6, Panel A. The base, or bottom pan 104 of the embodiment 200 may be similar to the base 104 illustrated in FIG. 5, Panel A. Base 104 may also include one or more offset circular vent holes 116 and 118 each having a filter and a retainer plate associated therewith as seen, for example, in detail in FIG. 5, Panel F. The structure of the first and/or second or additional sets of vent holes 202 and 204 may be similar to the structure of the set of vent holes 114 in the lid 102 of embodiment 100 as illustrated in FIG. 5, Panels A-F. Pull ring 218 may be connected to base 220 and is preferably located in the center of the concentric circles 202. Pull ring 218 may be attached by a rivet assembly 244a, 244b, and 244c as illustrated in exploded detail in FIG. 6, Panel D. Similarly, the second or additional sets of vent holes 204 may comprise four concentric circles having vent holes 204a, 204b, 204c and 204d which may be arranged around a pull ring 222 attached to a base 224 and connected to the lid 226 in the same manner as illustrated in FIG. 6, Panel D. The first set of vent holes 202 may be associated with a filter disk 206, a ring 208, and a perforated retainer plate 210 that snaps-on or screws-on and attaches to a post on the bottom side of the base plate 220 in the same manner that the post 146 of the embodiment 100 engages its perforated retainer plate 128 as illustrated in FIG. 5, Panel E. Referring to FIG. 6, Panel A, another filter disk 212 may be located under the second set of vent holes 204, and has an associated ring 214 and perforated retainer plate 216 below it. Perforated retainer plate 216 may also engage with, for example, a snap-on or screw-on post associated with pull ring 222 and base plate 224.

This alternative embodiment 200 also provides for improved circulation of the sterilant, such as steam, gas or gas plasma through the container so as to contact all the surgical instruments and the corners of the device, as discussed in conjunction with embodiment 100.

FIG. 8, Panels A-C depict a thermostatically controlled valve assembly 300 that may be of a wide variety of types, shapes or sizes, including, but not limited to, the type used in automotive radiator circulation systems or any other type of control valve capable of opening at a predetermined temperature and closing at a predetermined closing temperature. Thermostatic valve assembly 300 depicted in FIG. 8, Panels A-C is mounted on the filter retainer covering the opening 40 in filter retainer disc 32 so as to block sterilization medium flow into the container when thermostatic valve 310 is closed and allow the sterilization medium to flow into the container, or moisture to exit the container, when thermostatic valve 310 is open. Thermostatic valve 310 opens when the temperature of the inside of the container reaches a predetermined level allowing the sterilization medium to enter the container and thus sterilize the contents of the container. A very wide variety of thermostatic valve opening and closing temperatures may be used in the sterilization container of the present invention. The desired temperature range to activate thermostatic valve 310 may vary, depending upon, for example, the type of sterilization medium or sterilization processes being used. For example, if steam is the sterilizing medium, in one embodiment, the temperature range for the thermostatic valve operation may be between about 150 and about 225 degrees Fahrenheit. In embodiments in which gas or gas plasma is used, the temperature range for the thermostatic valve operation may be between about 100 and about 150 degrees Fahrenheit. Thermostatic valve 310 may be constructed such that it does not require disassembly in order to clean or sterilize, and its functionality may be tested by placing it in water at the preferred temperature range to observe the opening and closing of the valve. Additionally, thermostatic valve 310 may be made of copper, aluminum, or other materials, preferably metals, that conduct heat. Thermostatic valve 310 may also be plated or coated to prevent leaching of its material into the container. The thermostatic valve assembly may also contain a mechanism for recording the opening or closing of the valve, which may provide to the user recorded evidence of proper valve function. Any desired means for making such a record or evidence of valve function may be utilized with the sterilization container of the present invention. In one embodiment, a pin or marker perforates or marks a record material, such as paper or other fabric, when the valve opens. The marked record material may be observed and discarded or saved by the user as evidence of proper thermostatic valve function.

Thermostatic valve assembly 300 is mounted beneath opening 40, as shown in FIG. 8, Panel A in filter retainer 30. In one embodiment, the thermostatic valve regulates the flow of a sterilizing medium or moisture into and from the inside of the sterilization container. Thermostatic valve assembly 300 may include a thermostatic valve 310 and a cover 320 designed to act, together, as a means to seal the interior of the sterilization container from outside contaminants after completion of the sterilization process. Opening 40 extends through filter retainer 30, through middle ring 36, acting as a channel from opening 40 to filter retainer 30. FIG. 8, Panel C depicts a filter retainer adapted for a thermostatic valve assembly 300. In one embodiment, the thermostatic valve assembly is completely enclosed within the interior of the sterilization container.

Thermostatic valve 310 is, in one embodiment, held open by a temperature sensitive member prior to reaching a desired temperature. In one embodiment, the temperature sensitive member is made of a material that, for example, changes its physical characteristics, compresses, evaporates, softens or melts when the preferred temperature is reached, thereafter permitting the thermostatic valve to close at its closing temperature. After the temperature sensitive member is no longer able to hold the thermostatic valve open at its closing temperature, thermostatic valve 310 closes upon reaching its closing temperature, sealing the inside of the sterilization container from entry by contaminants, which may be present outside the container when the container is removed from the sterilization chamber. When the sterilization container is being prepared for a subsequent use, thermostatic valve 310 may be opened, providing a space for another temperature sensitive member to be inserted to hold the thermostatic valve open.

Valve retainer adapter 350, shown in FIG. 8, Panel D, may simply be placed about opening 40 in existing sterilization containers to adapt them for use in conjunction with a thermostatic valve. Adapter 350 may be made of one or more metal plates. In one embodiment, the adapter is made of one piece of material, such as metal. In another embodiment, the adapter is made of two pieces of material, one or more of which may be made from a metal plate (upper plate 355 and lower plate 360). Upper plate 355 and lower plate 360 may be circular with an inner and outer diameter, and may resemble a washer. Upper plate 355 and lower plate 360 may also have a plurality of holes 356 and 361, respectively, adapted to receive fasteners, such as for example, threaded fasteners. Upper plate 355 and lower plate 360 may be connected by a cylindrical connector 365, which is also preferably made of anodized aluminum or another metal that is thermoconductive.

In some embodiments utilizing a thermostatic valve assembly 300, a filter may not be necessary. Cover 320 and thermostatic valve 310 fit together so as to form a seal once the thermostatic valve closes, preventing contaminants from entering the interior of the sterilization container after the sterilization process. Cover 320 and corresponding valve 310 may be removed using the same latching or locking mechanism as the filter retention plate used in conjunction with an embodiment utilizing a filter.

Thermostatic valve assembly, in conjunction with other embodiments of the invention, may also be utilized to extend the length of time that its contents remain sterilized after the sterilization process. Use of thermostatic valve 310 and cover 320 prevents any contaminants from entering the container after it has been sterilized because thermostatic valve 310 closes when it reaches the desired closing temperature. Once thermostatic valve 310 closes and seals, the container will maintain its sterility and may be stored.

In one or more embodiments of the present invention, the material used to construct the sterilization container is a metal. In another embodiment of the present invention the sterilization container is constructed of aluminum. In another embodiment, the aluminum is an aluminum alloy, including, but not limited to, aluminum alloy 6061, which is commercially available from Alcoa, Inc. It may be desirable to perform additional processes on the metal, such as heat treatment, and in one embodiment, heat treatment is in a range from about T4 to about T6 temper. In another embodiment, the metal may be treated electrolytically, for example, in a bath containing an appropriate acid, such as sulfuric acid, to produce a uniform anodic coating on the metal surface. Coating thicknesses may be in the range of between about 0.1 and about 0.5 mils. Coating thicknesses in the range of between about 0.1 and about 0.5 mils typically result in improved sterilization characteristics in the container over thicker coatings. Coatings in the range of about 0.1 mils to about 0.5 mils are preferred. In one embodiment, the coating is within the range of about 0.2 to about 0.3 mils. The thickness of the anodizing in one embodiment should not exceed about 0.5 mils (0.0005 inches) and preferably should have a coating between about 0.2 mils. (0.0002 inches) and about 0.3 mils (0.0003 inches). This coating thickness is less than other coating used in the art, which are generally about 0.7 mils (0.0007 inches).

In a specific embodiment, all parts of the container are anodized after all manufacturing processes (metal forming, material removal, including welding and cutting) and material treatment (heat treatment, etc.). The anodic coating should not be applied to assemblies which will trap the electrolyte in joints or recesses.

Portions of assemblies or container that would be adversely affected by heat treatment or anodic coating should not be exposed to anodization unless such portions are protected by masking or electric insulation. Additionally, all aluminum parts to be anodized must be kept clean of all foreign substances such as grease, welding flux, and other metals such as iron or steel, which would acceleration corrosion of the aluminum alloy.

After applying a uniform anodic coating, all parts should be completely sealed in a known manner.

It is believed that one of ordinary skill in the art, using the preceding description, can utilize the present invention to the fullest extent.

It will be apparent to those skilled in the art that various modifications and variations can be made in the sterilization container system, apparatus and method of the present invention and its construction without departing from the scope and spirit of the invention. Other embodiments of the invention will be apparent to those skilled in the art from consideration of the specification and practice of the invention disclosed herein. It is intended that the specification and examples be considered as exemplary only of the present invention.

[image]

We claim:

1. A sterilization container comprising:
a lid having a first set of vent holes;
a filter means adjacent to said first set of vent holes; and
a bottom attachable to said lid, wherein said bottom comprises side walls, a base, and one or more sets of vent holes located in said base of said bottom;
wherein said sterilization container is adapted to provide passage for a sterilizing medium throughout the interior of said sterilization container.

2. The sterilization container of claim 1, wherein said sterilizing medium passes through said container via said first set of vent holes, and said one or more sets of vent holes located in said bottom.

3. The sterilization container of claim 1, wherein said sterilizing medium is selected from the group consisting of gas, gas plasma and steam.

4. The sterilization container of claim 1, wherein said container is metal.

5. The sterilization container of claim 1, further comprising a filter medium in fluid connection with said first set of vent holes, and said one or more sets of vent holes located in said base, wherein said sterilizing medium passes through said filter medium.

6. The sterilization container of claim 1, further comprising a second set of vent holes in said lid adapted to provide passage for a sterilization medium.

7. The sterilization container of claim 6, further comprising a filter medium in fluid connection with said second set of vent holes in said lid.

8. The sterilization container of claim 7, wherein said filter medium is selected from the group consisting of paper filter material, hydrophobic filter material, non-woven polypropylene polyolefin filter material, synthetic filter material, and a cellulosic filter material.

9. The sterilization container of claim 1, wherein said first set of vent holes in said lid, and said one or more sets of vent holes in said bottom each comprises a plurality of concentric holes.

10. The sterilization container of claim 6, wherein said second set of vent holes in said lid comprises a plurality of concentric holes.

11. The sterilization container of claim 5, wherein said filter medium is selected from the group consisting of paper filter material, hydrophobic filter material, non-woven polypropylene polyolefin filter material, synthetic filter material, and a cellulosic filter material.

12. The sterilization container of claim 1, further comprising:
one or more projections, channels, impressions or dimples in said lid; and
one or more projections, channels, impressions or dimples in said bottom,
wherein said projections, channels, impressions or dimples in said lid or said bottom are adapted to engage with other recessed dimples in other lids or bottoms.

13. The sterilization container of claim 12, wherein said dimples provide clearance for said one or more sets of vent holes in said bottom.

14. The sterilization container of claim 1, wherein said container prevents excess internal moisture by allowing evaporation.

15. The container of claim 1, wherein said container is stored containing sterile contents after sterilization.

16. The container of claim 1, wherein said container further comprises a means to insulate electrical contact between said lid and said bottom.

17. The container of claim 1, further comprising an instrument tray.

18. The container of claim 17, wherein said instrument tray comprises:
a base; and
one or more handles,
connected to said base wherein said base is perforated.

19. The container of claim 18, wherein said tray comprises two or more handles.

20. The container of claim 18, wherein said tray further comprises one or more side walls.

21. The container of claim 18, wherein said tray further comprises a divider system wherein said divider system is adapted to prevent contact between instruments in the tray.

22. The container of claim 21, wherein said divider system further comprises one or more instrument brackets.

23. The container of claim 18, wherein said instrument brackets are scalloped.

24. The container of claim 18, wherein said instrument tray has feet.

25. The container of claim 4, wherein said metal is selected from a group consisting of aluminum, aluminum alloy, and aluminum alloy 6061.

26. The container of claim 25, wherein said metal is heat-treated at a T temper in a range of between about T4 to about T6.

27. The container of claim 25, wherein said metal is an aluminum alloy that is anodized.

28. The container of claim 27, wherein said anodization produces a coating between about 0.1 mil and about 0.5 mil.

29. The container of claim 27, wherein said anodization produces a coating between about 0.2 mil and about 0.3 mil.

30. The container of claim 1, wherein said container maintains a sterile field after removal from a sterilization chamber.

31. The container of claim 1, wherein said container comprises an audible locking mechanism for attaching said lid to said bottom, and for attaching said filter to said vent holes, wherein said audible locking mechanism produces an audible signal when said locking mechanism is locked.

32. A sterilization container comprising:
a lid having a first set of vent holes;
a filter means adjacent to said first set of vent holes; and
a bottom attachable to said lid, wherein said bottom comprises side walls, a base, and at least a second and third set of vent holes located in said base of said bottom;
wherein said sterilization container is adapted to provide passage for a sterilizing medium throughout the interior of said sterilization container, wherein said sterilization container is made from an aluminum alloy.

33. The sterilization container of claim 32, wherein said sterilizing medium passes through said container via said first set of vent holes, said second set of vent holes, and said third set of vent holes.

34. The sterilization container of claim 32, wherein said sterilizing medium is selected from the group consisting of gas, gas plasma and steam.

35. The sterilization container of claim 32, further comprising a filter medium in fluid connection with said first set of vent holes, said second set of vent holes and said third set of vent holes, wherein said sterilizing medium passes through said filter medium.

36. The sterilization container of claim 32, further comprising a fourth set of vent holes adapted to provide passage for a sterilization medium.

37. The sterilization container of claim 36, further comprising a filter medium in fluid connection with said fourth set of vent holes.

38. The sterilization container of claim 37, wherein said filter medium is selected from the group consisting of paper filter material, hydrophobic filter material, non-woven polypropylene polyolefin filter material, synthetic filter material, and a cellulosic filter material.

39. The sterilization container of claim 32, wherein said first set of vent holes, said second set of vent holes, and said third set of vent holes each comprises a plurality of concentric holes.

40. The sterilization container of claim 36, wherein said fourth set of vent holes comprises a plurality of concentric holes.

41. The sterilization container of claim 35, wherein said filter medium is selected from the group consisting of paper filter material, hydrophobic filter material, non-woven polypropylene polyolefin filter material, synthetic filter material, and a cellulosic filter material.

42. The sterilization container of claim 32, further comprising:
one or more projections, channels, impressions or dimples in said lid; and
one or more projections, channels, impressions or dimples in said bottom,
wherein said projections, channels, impressions or dimples in said lid or said bottom are adapted to engage with other recessed dimples in other lids or bottoms for the purpose of stacking said container.

43. The sterilization container of claim 42, wherein said projections, channels, impressions or dimples provide clearance for said second and third set of vent holes.

44. The sterilization container of claim 32, wherein said container prevents excess moisture by allowing evaporation.

45. The container of claim 32, wherein said container is stored containing sterile contents after sterilization.

46. The container of claim 32, wherein said container further comprises a means to insulate electrical contact between said lid and said bottom.

47. The container of claim 32, further comprising an instrument tray.

48. The container of claim 47, wherein said instrument tray comprises:
a base; and
one or more handles connected to said base;
wherein said base is perforated.

49. The container of claim 48, wherein said tray further comprises two handles.

50. The container of claim 48, wherein said tray further comprises one or more side walls.

51. The container of claim 48, wherein said tray further comprises a divider systems wherein said divider system is adapted to prevent contact between multiple instruments.

52. The container of claim 51, wherein said divider system further comprises a plurality of instrument brackets.

53. The container of claim 51, wherein said instrument brackets are scalloped.

54. The container of claim 48, wherein said instrument tray has feet.

55. The container of claim 32, wherein said aluminum alloy is heat-treated.

56. The container of claim 55, wherein said heat-treatment is a T temper, wherein said T-temper is selected from the group consisting of T4, T5, and T6.

57. The container of claim 32, wherein said aluminum alloy is anodized.

58. The container of claim 57, wherein said anodization produces a coating between about 0.1 mil and about 0.5 mil.

59. The container of claim 57, wherein said anodization produces a coating between about 0.2 mil and about 0.3 mil.

60. The container of claim 32, wherein said container maintains a sterile field after removal from a sterilization chamber.

61. The container of claim 32, wherein said aluminum alloy is aluminum alloy 6061.

62. A sterilization container comprising:
a lid having a first set of vent holes;
a filter means adjacent to said first set of vent holes;
a bottom attachable to said lid, wherein said bottom comprises side walls, a base, and at least a second and third set of vent holes located in said base of said bottom; and
an instrument tray;
wherein said sterilization container is adapted to provide passage for a sterilizing medium throughout the interior of said sterilization container.

63. The sterilization container of claim 62, wherein said sterilizing medium passes through said container via said first set of vent holes, said second set of vent holes, and said third set of vent holes.

64. The sterilization container of claim 62, wherein said sterilizing medium is selected from the group consisting of gas, steam and gas plasma.

65. The sterilization container of claim 62, further comprising a filter medium in fluid connection with said first set of vent holes, said second set of vent holes and said third set of vent holes, wherein said sterilizing medium passes through said filter medium.

66. The sterilization container of claim 62, further comprising a fourth set of vent holes adapted to provide passage for a sterilization medium.

67. The sterilization container of claim 66, further comprising a filter medium in fluid connection with said fourth set of vent holes.

68. The sterilization container of claim 67, wherein said filter medium is selected from the group consisting of paper filter material, hydrophobic filter material, non-woven polypropylene polyolefin filter material, synthetic filter material, and a cellulosic filter material.

69. The sterilization container of claim 62, wherein said first set of vent holes, said second set of vent holes, and said third set of vent holes each comprises a plurality of concentric holes.

70. The sterilization container of claim 66, wherein said fourth set of vent holes comprises a plurality of concentric holes.

71. The sterilization container of claim 66, wherein said filter medium is selected from the group consisting of paper filter material, hydrophobic filter material, non-woven polypropylene polyolefin filter material, synthetic filter material, and a cellulosic filter material.

72. The sterilization container of claim 62, further comprising:
one or more projections, channels, impressions or dimples in said lid; and
one or more projections, channels, impressions or dimples in said bottom,
wherein said projections, channels, impressions or dimples in said lid or said bottom are adapted to engage with other recessed dimples in other lids or bottoms for the purpose of stacking said container.

73. The sterilization container of claim 72, wherein said projections, channels, impressions or dimples provide clearance for said second and third set of vent holes.

74. The sterilization container of claim 62, wherein said container prevents excess internal moisture by allowing evaporation.

75. The container of claim 62, wherein said container is made of a metal selected from the group consisting of aluminum, aluminum alloy, and aluminum alloy 6061.

76. The container of claim 62, wherein said container is stored containing sterile contents after sterilization.

77. The container of claim 62, wherein said container further comprises a means to insulate electrical contact between said lid and said bottom.

78. The container of claim 62, wherein said instrument tray comprises:
a base; and
one or more handles connected to said base;
wherein said base is perforated.

79. The container of claim 78, wherein said tray further comprises two or more handles.

80. The container of claim 78, wherein said tray further comprises one or more side walls.

81. The container of claim 78, wherein said tray further comprises a divider system wherein said divider system is adapted to prevent contact between instruments.

82. The container of claim 78, wherein said divider system further comprises a plurality of instrument brackets.

83. The container of claim 78, wherein said instrument brackets are scalloped.

84. The container of claim 78, wherein said instrument tray has feet.

85. The container of claim 62, wherein said container comprises a metal selected from the group consisting of aluminum, aluminum alloy, and aluminum alloy 6061.

86. The container of claim 85, wherein said metal is heat-treated.

87. The container of claim 86, wherein said heat-treatment is a T temper in the range of between about T4 to about T6.

88. The container of claim 85, wherein said metal is an aluminum alloy and said aluminum alloy is anodized.

89. The container of claim 88, wherein said anodization produces a coating between about 0.1 mil and about 0.5 mil.

90. The container of claim 88, wherein said anodization produces a coating between about 0.2 mil and about 0.3 mil.

91. The container of claim 62, wherein said container maintains a sterile field after removal from an autoclave.

92. The container of claim 62, wherein said container is adapted to be used for flash sterilization.

93. A sterilization container comprising:
a lid having a first set of vent holes;
a filter means adjacent to said first set of vent holes;
a thermostatic valve assembly in fluid connection with said lid and said interior of said sterilization container;
a bottom attachable to said lid, wherein said bottom comprises side walls, a base, and at least a second and third set of vent holes located in said base of said bottom; and
an instrument tray;
wherein said sterilization container is adapted to provide passage for a sterilizing medium throughout the interior of said sterilization container and wherein said container is made from an aluminum alloy.

94. The sterilization container of claim 93, wherein said sterilizing medium passes through said container via said first set of vent holes, said second set of vent holes, and said third set of vent holes.

95. The sterilization container of claim 93, wherein said thermostatic valve assembly comprises a recording mechanism, wherein said recording mechanism indicates openings of the valve.

96. The sterilization container of claim 95, wherein said thermostatic valve assembly is designed to open or close between about 100 and about 150 degrees F.

97. The sterilization container of claim 93, wherein said sterilizing medium is selected from a group consisting of gas, gas plasma and steam.

98. The sterilization container of claim 97, wherein said thermostatic valve assembly is designed to open or close between about 150 and about 225 degrees Fahrenheit.

99. The sterilization container of claim 93, further comprising a filter medium in fluid connection with said first set of vent holes, said second set of vent holes and said third set of vent holes, wherein said sterilizing medium passes through said filter medium.

100. The sterilization container of claim 93, further comprising a fourth set of vent holes adapted to provide passage for a sterilization medium.

101. The sterilization container of claim 100, further comprising a filter medium in fluid connection with said fourth set of vent holes.

102. The sterilization container of claim 101, wherein said filter medium is selected from the group consisting of: consisting of paper filter material, hydrophobic filter material, non-woven polypropylene polyolefin filter material, synthetic filter material, and a cellulosic filter material.

103. The sterilization container of claim 93, wherein said first set of vent holes, said second set of vent holes, and said third set of vent holes each comprises a plurality of concentric holes.

104. The sterilization container of claim 100, wherein said fourth set of vent holes comprises a plurality of concentric holes.

105. The sterilization container of claim 99, wherein said filter medium is selected from the group consisting of paper filter material, hydrophobic filter material, non-woven polypropylene polyolefin filter material, synthetic filter material, and a cellulosic filter material.

106. The sterilization container of claim 93, wherein said thermostatic valve assembly is housed entirely inside of said container.

107. The sterilization container of claim 93, further comprising:
one or more projections, channels, impressions or dimples in said lid; and
one or more projections, channels, impressions or dimples in said bottom,
wherein said projections, channels, impressions or dimples in said lid or said bottom are adapted to engage with other recessed dimples in other lids or bottoms for the purpose of stacking said container.

108. The sterilization container of claim 107, wherein said dimples provide clearance for said second and third set of vent holes.

109. The sterilization container of claim 93, wherein said container prevents excess internal moisture by allowing evaporation.

110. The sterilization container of claim 93, wherein said thermostatic valve assembly further comprises:
a cover adapted to completely cover said vent holes; and
a thermostatic valve adapted to provide a channel through said cover to the interior of said sterilization container;
wherein said cover and said thermostatic valve act together as a seal to said interior of said sterilization container.

111. The container of claim 93, wherein said thermostatic valve assembly comprises a recording mechanism, wherein said recording mechanism indicates opening of the valve.

112. The container of claim 93, wherein said container is stored containing sterile contents after sterilization.

113. The container of claim 93, wherein said container further comprises a means to insulate electrical contact between said lid and said bottom.

114. The container of claim 93, wherein said instrument tray comprises:
a base; and
one or more handles connected to said base;
wherein said base is perforated.

115. The container of claim 114, wherein said tray further comprises two handles.

116. The container of claim 114, wherein said tray further comprises one or more side walls.

117. The container of claim 114, wherein said tray further comprises a divider system wherein said divider system is adapted to prevent contact between multiple instruments.

118. The container of claim 117, wherein said divider system further comprises a plurality of instrument brackets.

119. The container of claim 117, wherein said instrument brackets are scalloped.

120. The container of claim 114, wherein said instrument tray has feet.

121. The container of claim 93, wherein said aluminum alloy is heat-treated.

122. The container of claim 121, wherein said heat-treatment is a T temper, wherein said T-temper is selected from the group consisting of T4, T5, and T6.

123. The container of claim 93, wherein said aluminum alloy is anodized.

124. The container of claim 123, wherein said anodization produces a coating between about 0.1 mil and about 0.5 mil.

125. The container of claim 123, wherein said anodization produces a coating between about 0.2 mil and about 0.3 mil.

126. The container of claim 93, wherein said container maintains a sterile field after removal from a sterilization chamber.

127. The container of claim 93, wherein said container is adapted to be used for flash sterilization.

128. A sterilization container comprising:
a lid having a first set of vent holes;
a filter means adjacent to said first set of vent holes;
a thermostatic valve assembly in fluid connection with said lid and said interior of said sterilization container;
a bottom attachable to said lid, wherein said bottom comprises side walls, a base, and at one or more sets of vent holes located in said base of said bottom; and
an instrument tray;
wherein said sterilization container is adapted to provide passage for a sterilizing medium throughout the interior of said sterilization container and wherein said container is made from an aluminum alloy.

129. The sterilization container of claim 128, wherein said sterilizing medium passes through said container via said first set of vent holes, and said one or more sets of vent holes located in said bottom.

130. The sterilization container of claim 128, wherein said sterilizing medium is selected from the group consisting of gas, gas plasma and steam.

131. The sterilization container of claim 130, wherein said thermostatic valve assembly is designed to open or close between about 100 and about 150 degrees F.

132. The sterilization container of claim 128, wherein said sterilizing medium comprises steam.

133. The sterilization container of claim 132, wherein said thermostatic valve assembly is designed to open or close between about 150 and about 225 degrees Fahrenheit.

134. The sterilization container of claim 128, further comprising a filter medium in fluid connection with said first set of vent holes, and said one or more sets of vent holes in said bottom, wherein said sterilizing medium passes through said filter medium.

135. The sterilization container of claim 128, further comprising a second set of vent holes in said lid adapted to provide passage for a sterilization medium.

136. The sterilization container of claim 135, further comprising a filter medium in fluid connection with said second set of vent holes in said lid.

137. The sterilization container of claim 136, wherein said filter medium is selected from the group consisting of paper filter material, hydrophobic filter material, non-woven polypropylene polyolefin filter material, synthetic filter material, and a cellulosic filter material.

138. The sterilization container of claim 128, wherein said first set of vent holes, and said one or more sets of vent holes in said bottom each comprises a plurality of concentric holes.

139. The sterilization container of claim 136, wherein said second said of vent holes in said lid comprises a plurality of concentric holes.

140. The sterilization container of claim 134, wherein said filter medium is selected from the group consisting of paper filter material, hydrophobic filter material, non-woven polypropylene polyolefin filter material, synthetic filter material, and a cellulosic filter material.

141. The sterilization container of claim 128, wherein said thermostatic valve assembly is housed entirely inside of said container.

142. The sterilization container of claim 128, further comprising:
one or more projections, channels, impressions or dimples in said lid; and
one or more projections, channels, impressions or dimples in said bottom,
wherein said projections, channels, impressions or dimples in said lid or said bottom are adapted to engage with other recessed dimples in other lids or bottoms for the purpose of stacking said container.

143. The sterilization container of claim 142, wherein said projections, channels, impressions or dimples provide clearance for said one or more sets of vent holes in said bottom.

144. The sterilization container of claim 128, wherein said container prevents excess internal moisture by allowing evaporation.

145. The sterilization container of claim 128, wherein said thermostatic valve assembly further comprises:
a cover adapted to completely cover said vent holes; and
a thermostatic valve adapted to provide a channel through said cover to the interior of said sterilization container;
wherein said cover and said thermostatic valve act together as a seal to said interior of said sterilization container.

146. The container of claim 128, wherein said thermostatic valve assembly comprises a recording mechanism, wherein said recording mechanism indicates opening of the valve.

147. The container of claim 128, wherein said container is stored containing sterile contents after sterilization.

148. The container of claim 128, wherein said container further comprises a means to insulate electrical contact between said lid and said bottom.

149. The container of claim 128, wherein said instrument tray comprises:
a base; and
one or more handles connected to said base;
wherein said base is perforated.

150. The container of claim 149, wherein said tray further comprises two handles.

151. The container of claim 149, wherein said tray further comprises one or more side walls.

152. The container of claim 149, wherein said tray further comprises a divider system wherein said divider system is adapted to prevent contact between multiple instruments.

153. The container of claim 152, wherein said divider system further comprises a plurality of instrument brackets.

154. The container of claim 152, wherein said instrument brackets are scalloped.

155. The container of claim 149, wherein said instrument tray has feet.

156. The container of claim 128, wherein said aluminum alloy is heat-treated.

157. The container of claim 156, wherein said heat-treatment is a T temper, wherein said T-temper is selected from the group consisting of T4, T5, and T6.

158. The container of claim 128, wherein said aluminum alloy is anodized.

159. The container of claim 158, wherein said anodization produces a coating between about 0.1 mil and about 0.5 mil.

160. The container of claim 158, wherein said anodization produces a coating between about 0.2 mil and about 0.3 mil.

161. The container of claim 128, wherein said container maintains a sterile field after removal from an autoclave.

162. The container of claim 128, wherein said container is adapted to be used for flash sterilization.

163. A method for sterilizing items comprising the steps of:
a) placing the items to be sterilized in a container apparatus, said container apparatus comprising:
a lid having a first set of vent holes;
a filter means adjacent to said first set of vent holes; and
a bottom attachable to said lid, wherein said bottom comprises side walls,
a base, and one or more sets of vent holes located in said base of said bottom;
wherein said sterilization container is adapted to provide passage for a sterilizing medium throughout the interior of said sterilization container.
b) passing a sterilizing medium through said first set of vent holes, around the inside of said container and out of said container; and
c) removing said sterilization container from an autoclave.

164. The method of claim 163, wherein said sterilizing medium passes out of said container through said at least one or more sets of vent holes in said bottom.

165. The method of claim 163, wherein said sterilizing medium is selected from the group consisting of gas plasma and steam.

166. The method of claim 163, further comprising a thermostatic valve assembly.

167. The method of claim 166, wherein said thermostatic valve assembly is designed to open or close between about 100 and about 150 degrees F when gas plasma is used as the sterilizing medium.

168. The method of claim 166, wherein said thermostatic valve assembly is designed to open or close between about 150 and about 225 degrees F when steam is used as the sterilizing medium.

169. The method of claim 163, further comprising a filter medium in fluid connection with said first set of vent holes, and said one or more sets of vent holes in said bottom.

170. The method of claim 163, further comprising a second set of vent holes in said lid to provide passage for a sterilization medium.

171. The method of claim 170, further comprising a filter medium in fluid connection with said second set of vent holes in said lid.

172. The method of claim 171, wherein said filter medium is selected from the group consisting of paper filter material, hydrophobic filter material, non-woven polypropylene polyolefin filter material, synthetic filter material, and a cellulosic filter material.

173. The method of claim 163, wherein said first set of vent holes, and said one or more sets of vent holes in said bottom comprises a plurality of concentric holes.

174. The method of claim 170, wherein said second set of vent holes in said lid comprises a plurality of concentric holes.

175. The method of claim 169, wherein said filter medium is selected from the group consisting of paper filter material, hydrophobic filter material, non-woven polypropylene polyolefin filter material, synthetic filter material, and a cellulosic filter material.

176. The method of claim 166, wherein said thermostatic valve assembly is housed entirely inside of said container.

177. The method of claim 163, wherein said sterilization container further comprises:
one or more projections, channels, impressions or dimples in said lid; and
one or more projections, channels, impressions or dimples in said bottom,
wherein said projections, channels, impressions or dimples in said lid or said bottom are adapted to engage with other recessed dimples in other lids or bottoms for the purpose of stacking said container.

178. The method of claim 177, wherein said projections, channels, impressions or dimples provide clearance for said second and third set of vent holes.

179. The method of claim 163, wherein said sterilization container prevents excess internal moisture by allowing evaporation.

180. The method of claim 166, wherein said thermostatic valve assembly further comprises:
a cover adapted to completely cover said vent holes; and
a thermostatic valve adapted to provide a channel through said cover to the interior of said sterilization container,
wherein said cover and said thermostatic valve act together as a seal to said interior of said sterilization container.

181. The method of claim 163, further comprising storing said container containing sterile contents after sterilization.

182. The method of claim 163, further comprising providing a means to insulate electrical contact between said lid and said bottom.

183. The method of claim 163, further comprising an instrument tray.

184. The method of claim 183, wherein said instrument tray further comprises:
a base; and
one or more handles connected to said base;
wherein said base is perforated.

185. The method of claim 184, wherein said tray further comprises two handles.

186. The method of claim 184, wherein said tray further comprises one or more side walls.

187. The method of claim 184, wherein said tray further comprises a divider system wherein said divider system is adapted to prevent contact between multiple instruments.

188. The method of claim 187, wherein said divider system further comprises a plurality of instrument brackets.

189. The method of claim 187, wherein said instrument brackets are scalloped.

190. The method of claim 184, wherein said instrument tray has feet.

191. The method of claim 163, wherein said container further comprises an aluminum alloy.

192. The method of claim 191, further comprising heat treating said aluminum alloy.

193. The method of claim 192, wherein said heat-treatment is a T temper, wherein said T-temper is selected from the group consisting of T4, T5, and T6.

194. The method of claim 192, further comprising anodizing said aluminum alloy.

195. The method of claim 194, wherein said anodization produces a coating between about 0.1 mil and about 0.5 mil.

196. The method of claim 194, wherein said anodization produces a coating between about 0.2 mil and about 0.3 mil.

197. The method of claim 163, further comprising maintaining a sterile field after said removing step.

198. The method of claim 163, wherein said method for sterilization is a method for flash sterilization.

199. A sterilization container according to claim 32, 62, 93 or 128 further comprising an audible locking mechanism for attaching said lid to said bottom, and for attaching said filter to said vent holes, wherein said audible locking mechanism produces an audible signal when said locking mechanism is locked.
